(19) United States
(12) Patent Application Publication
(10) Pub. No.: US 2021/0196725 A1
(43) Pub. Date: Jul. 1, 2021

(54) TREATMENT OF RELAPSED FOLLICULAR LYMPHOMA

(71) Applicant: MEI Pharma, Inc., San Diego, CA (US)

(72) Inventor: Daniel P. GOLD, San Diego, CA (US)

(21) Appl. No.: 17/268,050

(22) PCT Filed: Aug. 13, 2019
(86) PCT No.: PCT/US2019/046408

§ 371 Date: Feb. 11, 2021

Related U.S. Application Data
(60) Provisional application No. 62/836,507, filed on Apr. 19, 2019.
(60) Provisional application No. 62/718,926, filed on Aug. 14, 2018.

(51) Int. Cl. A61K 31/5377; C07K 16/28; A61P 35/00; A61K 45/06

(52) U.S. Cl. CPC A61K 31/5377; C07K 16/2887; C07K 2317/76; A61K 45/06; A61P 35/00

(57) ABSTRACT

Provided herein are methods treating follicular lymphoma (FL) in subjects having early disease progression after immunochemotherapy using a phosphoinositide-3-kinase (PI3K) inhibitor. In certain embodiments, the methods comprise treating FL in subjects having disease progression within 24 months of initiating first-line or subsequent immunochemotherapy using a phosphoinositide-3-kinase (PI3K) inhibitor.

### CROSS REFERENCE TO RELATED APPLICATIONS

This application claims the benefit of U.S. Provisional Application No. 62/718,926, filed Aug. 14, 2018 and U.S. Provisional Application No. 62/836,507, filed Apr. 19, 2019; the disclosure of each of the prior applications is considered part of, and is incorporated by reference in, the disclosure of this application.

### BACKGROUND OF THE DISCLOSURE

Follicular lymphoma (FL) is among the most common malignant lymphomas worldwide and remains incurable for most patients.

### SUMMARY OF THE DISCLOSURE

Some embodiments provided herein describe a method of treating follicular lymphoma (FL), comprising administering to a subject in need thereof a therapeutically effective amount of a compound of Formula (I):

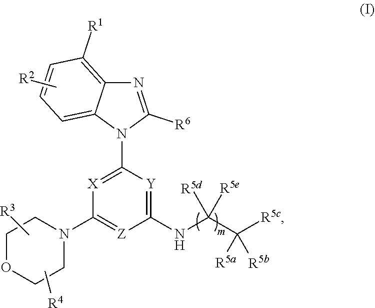

or an enantiomer, a mixture of enantiomers, a mixture of two or more diastereomers, or an isotopic variant thereof; or a pharmaceutically acceptable salt, solvate, or hydrate; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro;
   (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen;
      (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{x}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a},
   —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein the subject has progression of disease within 24 months of initiating treatment of FL with immunochemotherapy (POD24) or the subject has relapsed/refractory FL.

In some embodiments of the methods provided herein, R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments of the methods provided herein, R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments of the methods provided herein, R^{5a} and R^{5b} are each methyl, optionally substituted with one, two, or three halo(s).

In some embodiments of the methods provided herein, nisi.

In some embodiments of the methods provided herein, R^{5f} and R^{5g} are each hydrogen.

In some embodiments of the methods provided herein, n is 0.

In some embodiments of the methods provided herein, m is 0.

In some embodiments of the methods provided herein, the compound of Formula (I) is of Formula (XI):

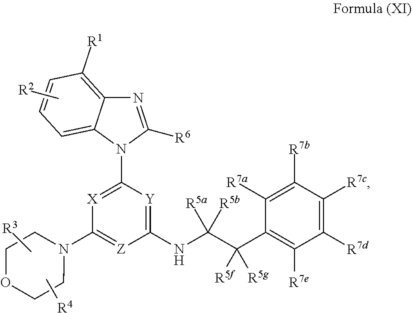

or an enantiomer, a mixture of enantiomers, a mixture of two or more diastereomers, or an isotopic variant thereof; or a pharmaceutically acceptable salt, solvate, hydrate, or prodrug thereof; wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or
two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

In some embodiments of the methods provided herein:
X, Y, and Z are each N;
R¹ and R² are each hydrogen;
R³ and R⁴ are each hydrogen;
R^{5a} is C₁₋₆ alkyl;
R^{5b} is C₁₋₆ alkyl;
R^{5c} is —(CH₂)-phenyl, wherein R^{5c} is optionally substituted with one, two, three, or four substituents
Q;
R^{5d} and R^{5e} are each hydrogen;
R⁶ is CHF₂;
m is 0; and
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

In some embodiments of the methods provided herein, R^{5a} and R^{5b} are each methyl, optionally substituted with one or more halos.

In some embodiments of the methods provided herein, the compound of Formula (I) is Compound A35:

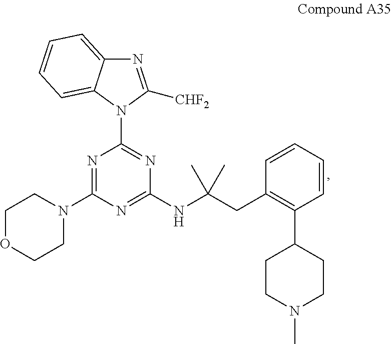 [SMILES: CN1CCC(c2ccccc2CC(C)(C)Nc2nc(N3CCOCC3)nc(-n3c(C(F)F)nc4ccccc43)n2)CC1]

or an isotopic variant thereof, a pharmaceutically acceptable salt, solvate, hydrate, or prodrug thereof.

In some embodiments of the methods provided herein, the compound of Formula (I) is Compound A36:

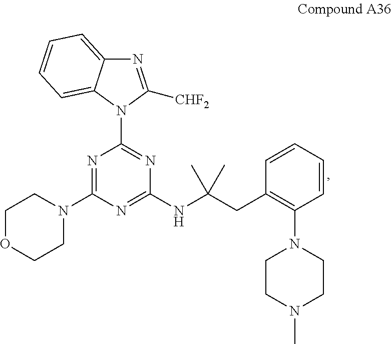 [SMILES: CN1CCN(c2ccccc2CC(C)(C)Nc2nc(N3CCOCC3)nc(-n3c(C(F)F)nc4ccccc43)n2)CC1]

or an isotopic variant thereof, a pharmaceutically acceptable salt, solvate, hydrate, or prodrug thereof.

In some embodiments of the methods provided herein, the compound of Formula (I) is Compound A68:

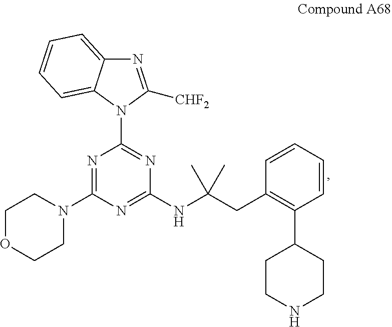 [SMILES: CC(C)(Cc1ccccc1C1CCNCC1)Nc1nc(N2CCOCC2)nc(-n2c(C(F)F)nc3ccccc32)n1]

or an isotopic variant thereof, a pharmaceutically acceptable salt, solvate, hydrate, or prodrug thereof.

In some embodiments of the methods provided herein, the compound of Formula (I) is Compound A70:

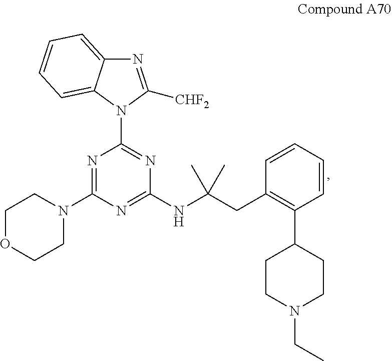 [SMILES: CCN1CCC(c2ccccc2CC(C)(C)Nc2nc(N3CCOCC3)nc(-n3c(C(F)F)nc4ccccc43)n2)CC1]

or an isotopic variant thereof, a pharmaceutically acceptable salt, solvate, hydrate, or prodrug thereof.

In some embodiments of the methods provided herein, the compound of Formula (I) is Compound A37:

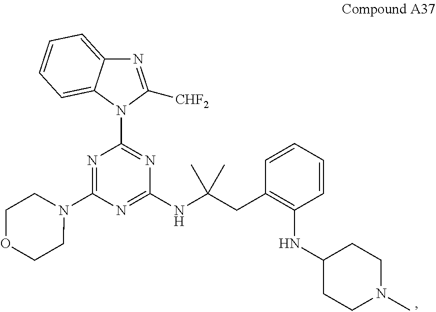 [SMILES: CN1CCC(Nc2ccccc2CC(C)(C)Nc2nc(N3CCOCC3)nc(-n3c(C(F)F)nc4ccccc43)n2)CC1]

or an isotopic variant thereof, a pharmaceutically acceptable salt, solvate, hydrate, or prodrug thereof.

In some embodiments of the methods provided herein, the compound of Formula (I) is Compound A38:

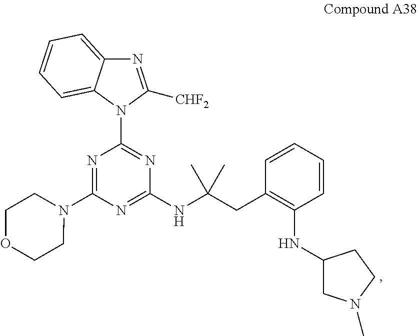 [SMILES: CN1CCC(Nc2ccccc2CC(C)(C)Nc2nc(N3CCOCC3)nc(-n3c(C(F)F)nc4ccccc43)n2)C1]

or an isotopic variant thereof, a pharmaceutically acceptable salt, solvate, hydrate, or prodrug thereof.

In some embodiments of the methods provided herein, the compound of Formula (I) is Compound A41:

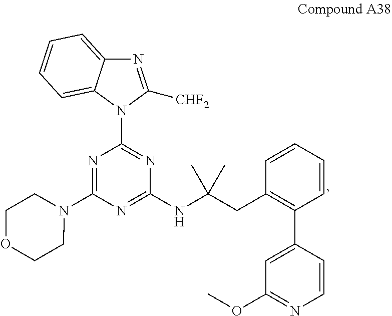 [SMILES: COc1cc(-c2ccccc2CC(C)(C)Nc2nc(N3CCOCC3)nc(-n3c(C(F)F)nc4ccccc43)n2)ccn1]

or an isotopic variant thereof, a pharmaceutically acceptable salt, solvate, hydrate, or prodrug thereof.

In some embodiments of the methods provided herein, the compound of Formula (I) is Compound A42:

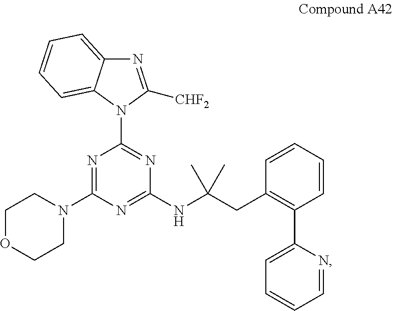 [SMILES: CC(C)(Cc1ccccc1-c1ccccn1)Nc1nc(N2CCOCC2)nc(-n2c(C(F)F)nc3ccccc32)n1]

or an isotopic variant thereof, a pharmaceutically acceptable salt, solvate, hydrate, or prodrug thereof.

In some embodiments of the methods provided herein, the compound of Formula (I) is Compound A43:

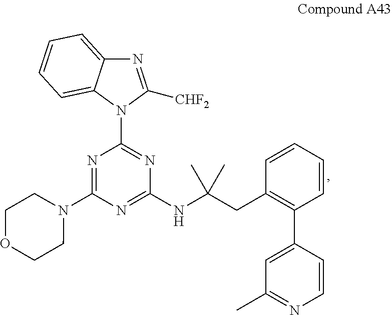 [SMILES: Cc1cc(-c2ccccc2CC(C)(C)Nc2nc(N3CCOCC3)nc(-n3c(C(F)F)nc4ccccc43)n2)ccn1]

or an isotopic variant thereof, a pharmaceutically acceptable salt, solvate, hydrate, or prodrug thereof.

In some embodiments of the methods provided herein, the compound of Formula (I) is Compound A44:

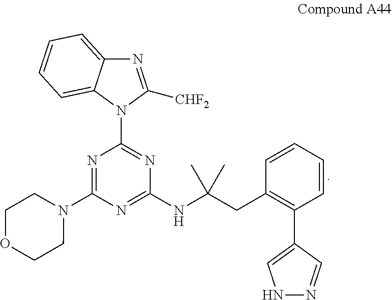 [SMILES: CC(C)(Cc1ccccc1-c1cn[nH]c1)Nc1nc(N2CCOCC2)nc(-n2c(C(F)F)nc3ccccc32)n1]

or an isotopic variant thereof, a pharmaceutically acceptable salt, solvate, hydrate, or prodrug thereof.

In some embodiments of the methods provided herein, about 30 mg, about 60 mg, about 120 mg, or about 180 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

In some embodiments of the methods provided herein, about 60 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject orally.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is formulated as a tablet or capsule.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject daily.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day, twice per day, or three times per day.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day.

In some embodiments of the methods provided herein, about 60 mg/day of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

In some embodiments of the methods provided herein, the subject has progression of disease within 24 months of initiating treatment of FL with first-line immunochemotherapy.

In some embodiments of the methods provided herein, the immunochemotherapy comprises administering to the subject a combination of chemotherapy and immunotherapy agents selected from i) bendamustine and rituximab; ii) RCHOP (rituximab, cyclophosphamide, doxorubicin hydrochloride, vincristine sulfate, and prednisone); iii) RCVP (rituximab, cyclophosphamide, vincristine sulfate, prednisone); iv) FCMR (fludarabine, cyclophosphamide, mitoxantrone and rituximab); v) fludarabine+rituximab; and vi) RFND (rituximab, fludarabine, mitoxantrone, dexamethasone).

In some embodiments of the methods provided herein, the immunochemotherapy comprises administering to the subject R-CHOP (rituximab, cyclophosphamide, doxorubicin hydrochloride, vincristine sulfate, and prednisone).

In some embodiments of the methods provided herein, the immunochemotherapy further comprises administering to the subject a BTK inhibitor

In some embodiments of the methods provided herein, the method comprises a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle.

In some embodiments of the methods provided herein, the CS is continued until at least one incidence of intolerable toxicity occurs/is observed.

In some embodiments of the methods provided herein, the at least one toxicity is enterocolitis, a cutaneous toxicity, liver toxicity, pulmonary toxicity, infection, or any combination thereof.

In some embodiments of the methods provided herein, the method comprises at least three 28-day cycles, wherein:
(i) the first two cycles comprise a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle; and
(ii) the third and subsequent cycles comprise an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle.

In some embodiments of the methods provided herein, T-cells are recovered and/or re-populated during the 21 days without treatment.

In some embodiments of the methods provided herein, regulatory T-cells (TREG) and/or effector T-cells are recovered and/or re-populated during the 21 days without treatment.

In some embodiments of the methods provided herein, the incidence of at least one toxicity is reduced.

In some embodiments of the methods provided herein, the at least one toxicity is enterocolitis, a cutaneous toxicity, liver toxicity, pulmonary toxicity, infection, or any combination thereof.

In some embodiments of the methods provided herein, the IS is continued until progression of disease.

In some embodiments of the methods provided herein, the subject has progression of disease within 24 months (POD24) of initiating treatment of FL with first-line immunochemotherapy. In some embodiments, the first-line immunochemotherapy comprises administering to the subject R-CHOP (rituximab, cyclophosphamide, doxorubicin hydrochloride, vincristine sulfate, and prednisone), G-CHOP (Gazyza, cyclophosphamide, doxorubicin hydrochloride, vincristine sulfate, and prednisone), RB (rituximab, bendamustine), R-CVP (rituximab, cyclophosphamide, vincristine sulfate, and prednisone), or similar regimens.

In some embodiments of the methods provided herein, an additional therapeutic agent is administered in combination with the compound of Formula (I), or an enantiomer, a mixture of enantiomers, a mixture of two or more diastereomers, or an isotopic variant thereof; or a pharmaceutically acceptable salt, solvate, hydrate, or prodrug thereof. In some embodiments of the methods provided herein, the additional therapeutic agent is rituximab. In some embodiments of the methods provided herein, rituximab is administered at a dose of about 375 mg/m². In some embodiments of the methods provided herein, 8 doses of rituximab are administered to the subject over a period of about 6 months the subject is treated for a period of about 6 months.

In an aspect, provided herein is a composition, comprising:

(i) a compound of Formula (I):

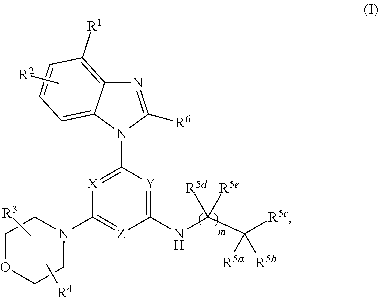

or an enantiomer, a mixture of enantiomers, a mixture of two or more diastereomers, or an isotopic variant thereof; or a pharmaceutically acceptable salt, solvate, or hydrate; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro;
   (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen;
   (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a},
   —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl; and
(ii) rituximab, cyclophosphamide, doxorubicin hydrochloride, vincristine sulfate, prednisone, gazyza, fludarabine, mitoxantrone, mitoxantrone, or dexamethasone.

In another aspect, provided herein is a pharmaceutical composition, comprising:
(i) a compound of Formula (I):

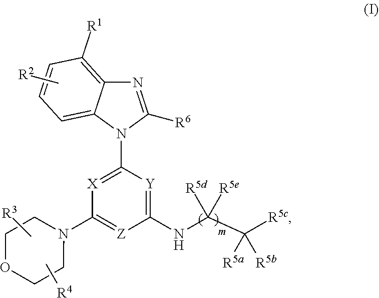

or an enantiomer, a mixture of enantiomers, a mixture of two or more diastereomers, or an isotopic variant thereof; or a pharmaceutically acceptable salt, solvate, or hydrate; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro;
   (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen;
   (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a},
   —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b)
C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
(ii) rituximab, cyclophosphamide, doxorubicin hydrochloride, vincristine sulfate, prednisone, gazyza, fludarabine, mitoxantrone, mitoxantrone, or dexamethasone; and
(iii) at least one pharmaceutically acceptable excipient or pharmaceutically acceptable carrier.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1B. Schematic representations of dosing schedules: A depicts the Continuous Schedule (CS); and B depicts the Intermittent Schedule (IS).

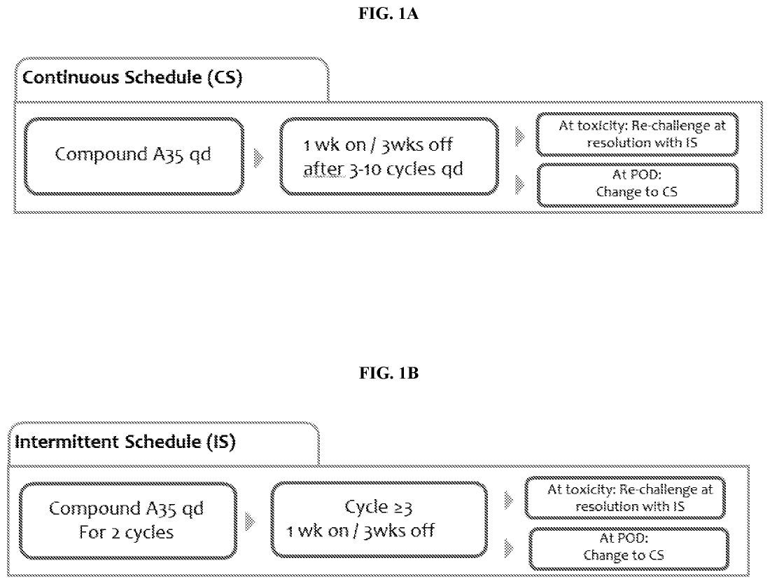

FIG. 2. Graphical representation showing the best change from baseline of measurable lesions for treatment with Compound A35 alone or in combination with rituximab on a continuous dosing schedule (CS) or intermittent dosing schedule (IS) in follicular lymphoma patients (N=49).

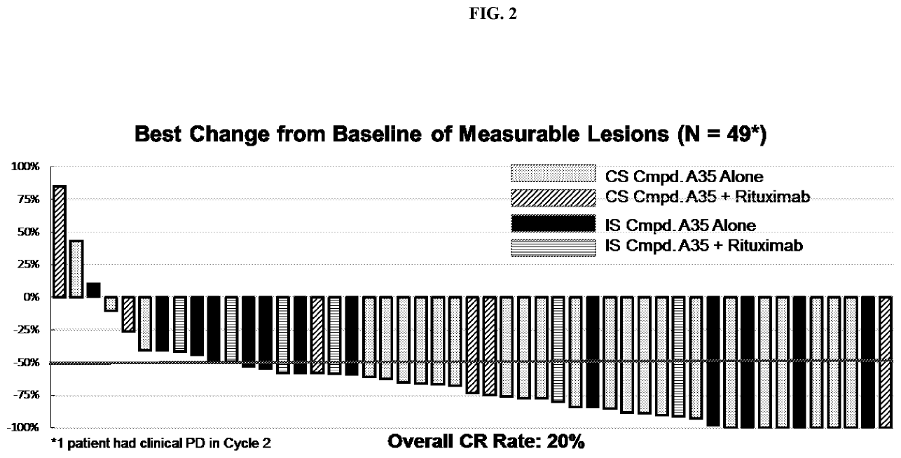

FIG. 3. Graphical representation showing preferential tumor exposure of Compound A35 over a 4-hour and 24-hour period.

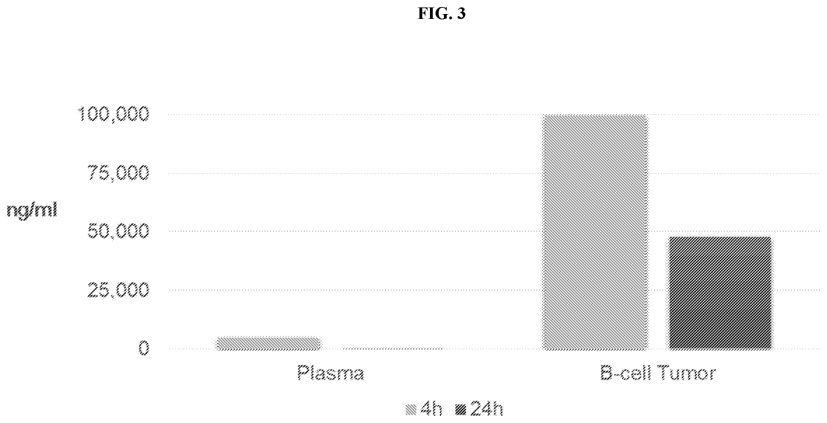

FIG. 4. Graphical representation showing preferential retention of Compound A35 compared to idelalisib in murine B-cell tumors.

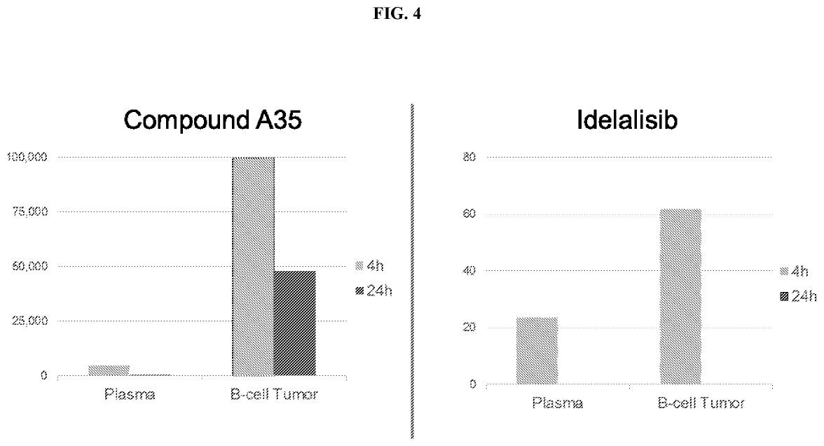

FIG. 5 Schematic representation of monotherapy treatment paradigm with Compound A35 in patients with R/R FL.

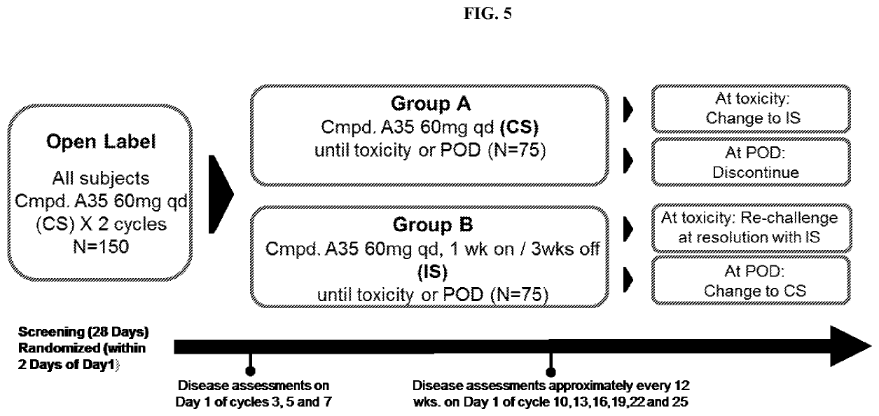

FIGS. 6A-6B. Graphical representation of Intermittent Dosing Schedule with Compound A35 (A) compared to parsaclisib (B) to maintain disease control.

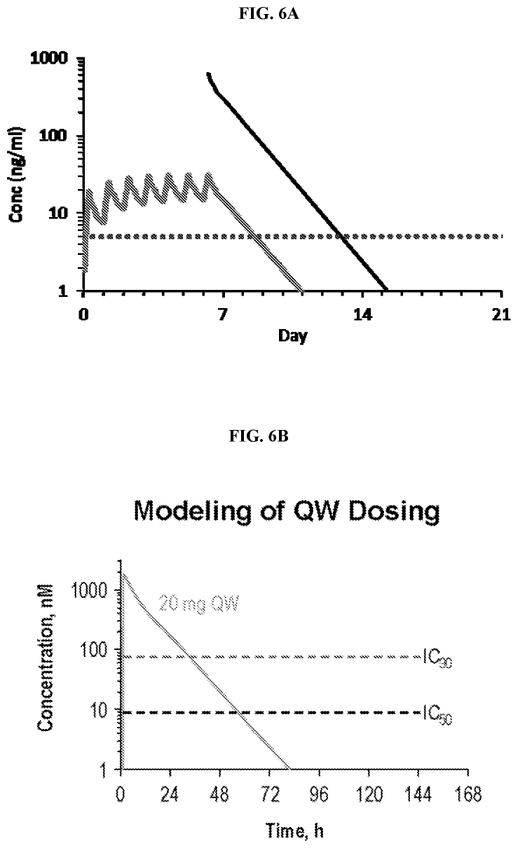

### DETAILED DESCRIPTION OF THE INVENTION

Some embodiments provided herein describe pharmaceutical compositions and methods for treating patients with follicular lymphoma who have early disease progression after immuochemotherapy. In some instances, the patients have early disease progression after first-line therapy with immunochemotherapy.

Large observational studies and clinical study databases have identified a subset of patients with follicular lymphoma (FL) with particularly poor long-term outcome despite initial therapy with standard immunochemotherapy. Specifically, in the National LymphoCare Study, patients with FL whose disease has progressed within 24 months of first-line R-CHOP therapy, designated POD24, were found to have a 5-year overall survival of 50% compared to 90% in the reference group of patients who were not POD24 (Casulo 2015). This trend was maintained after adjustment for FL International Prognostic Index (hazard ratio, 6.44; 95% CI, 4.33 to 9.58) and confirmed in a validation cohort.

Poor survival was also reported in the subset of patients with FL enrolled on the University of Iowa/Mayo Clinic Lymphoma SPORE Molecular Epidemiology Resource (MER) with early events (i.e., progression of disease) after first line immune-chemotherapy resulting in event-free survival (EFS) of 12 months (EFS12) or 24 months (EFS24), which was then confirmed in a validation cohort from 2 hospitals in Lyon, France (Maurer 2016).

It is estimated that approximately 20% of patients with FL treated with initial anti-CD20-based immunochemotherapy have early disease progression (i.e., qualify as POD24 or EFS24), but there are no clear methods to identify this subset of patients based on pre-treatment characteristics. Therefore, the characterization of this subset of FL patients with poor long-term survival remains a clinical definition and is based on the observation of disease progression within 24 months of completing initial chemoimmunotherapy. The data indicates that this subset of FL patients are uniquely at risk with different disease biology and and may benefit from alternate therapies, either up front or at the time of relapse.

Patients with early disease progression are typically retreated with second line chemoimmunotherapy, often using an alternate chemotherapy regimen with an anti-CD20 antibody, but results are usually insufficient in most patients as documented by a median overall survival <5 year. Lymphoma experts have indicated the response rates in patients with POD24 receiving standard therapy is approximately 50% and the median progression free survival is approximately 1 year. Alternate treatment strategies, including drugs with different mechanism(s) of action, are needed for this patient population. Provided herein in some embodiments are pharmaceutical compositions, comprising a therapeutic agent with a different mechanism of action compared to conventional FL therapy, and methods for treating this group of patients who are uniquely at risk, wherein the treatment provides a high response rate. In some embodiments, the therapeutic agent useful for treating this patient group is a PI3K delta inhibitor, alone or in combination with a CD20 inhibitor.

### Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “therapeutically effective amount” or “effective amount” are meant to include the amount of a compound that, when administered, is sufficient to prevent development of, or alleviate to some extent, one or more of the symptoms of the disorder, disease, or condition being treated. The terms “therapeutically effective amount” or “effective amount” also refer to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson Ed., CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a disorder, disease, or condition.

The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” refers to a material that is not found in nature or that has been structurally modified or synthesized by man.

The term “PI3K” refers to a phosphoinositide 3-kinase or variant thereof, which is capable of phosphorylating the inositol ring of PI in the D-3 position. The term “PI3K variant” is intended to include proteins substantially homologous to a native PI3K, i.e., proteins having one or more naturally or non-naturally occurring amino acid deletions, insertions, or substitutions (e.g., PI3K derivatives, homologs, and fragments), as compared to the amino acid sequence of a native PI3K. The amino acid sequence of a PI3K variant is at least about 80% identical, at least about 90% identical, or at least about 95% identical to a native PI3K. Examples of PI3K include, but are not limited to, p110α, p110β, p110δ, p110γ, PI3K-C2α, PI3K-C2β, PI3K-C2γ, Vps34, mTOR, ATM, ATR, and DNA-PK. See, Fry, Biochem. Biophys. Acta 1994, 1226, 237-268; Vanhaesebroeck and Waterfield, Exp. Cell. Res. 1999, 253, 239-254; and Fry, Breast Cancer Res. 2001, 3, 304-312. PI3Ks are classified into at least four classes. Class I includes p110α, p110β, p110δ, and p110γ. Class II includes PI3K-C2α, PI3K-C2β, and PI3K-C2γ. Class III includes Vps34. Class IV includes mTOR, ATM, ATR, and DNA-PK. In certain embodiments, the PI3K is a Class I kinase. In certain embodiments, the PI3K is p110α, p110β, p110δ, or p110γ. In certain embodiments, the PI3K is PI3K delta. In certain embodiments, the PI3K is a variant of a Class I kinase. In certain embodiments, the PI3K is a p110α mutant. Examples of p110α mutants include, but are not limited to, R38H, G106V, K111N, K227E, N345K, C420R, P539R, E542K, E545A, E545G, E545K, Q546K, Q546P, E453Q, H710P, I800L, T1025S, M10431, M1043V, H1047L, H1047R, and H1047Y (Ikenoue et al., Cancer Res. 2005, 65, 4562-4567; Gymnopoulos et al., Proc. Natl. Acad Sci., 2007, 104, 5569-5574). In certain embodiments, the PI3K is a Class II kinase. In certain embodiments, the PI3K is PI3K-C2α, PI3K-C2β, or PI3K-C2γ. In certain embodiments, the PI3K is a Class III kinase. In certain embodiments, the PI3K is Vps34. In certain embodiments, the PI3K is a Class IV kinase. In certain embodiments, the PI3K is mTOR, ATM, ATR, or DNA-PK.

The term “CD20” refers to an activated-glycosylated phosphoprotein expressed on the surface of all B-cells beginning at the pro-B phase (CD45R+, CD117+) and progressively increasing in concentration until maturity. The CD20 protein in humans is encoded by the MS4A1 gene. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes a B-lymphocyte surface molecule that plays a role in the development and differentiation of B-cells into plasma cells. This family member is localized to 11q12, among a cluster of family members. Alternative splicing of this gene results in two transcript variants that encode the same protein. The protein has no known natural ligand and its function is to enable optimal B-cell immune response, specifically against T-independent antigens. It is suspected that it acts as a calcium channel in the cell membrane. It has been shown that CD20 plays a role in the microenvironmental interactions of B cells and are therefore used to treat some types of cancer.

The term “antibody” refers to (a) immunoglobulin polypeptides and immunologically active portions of immunoglobulin polypeptides, i.e., polypeptides of the immunoglobulin family, or fragments thereof, that contain an antigen binding site that specifically binds to a specific antigen, or (b) conservatively substituted derivatives of such immunoglobulin polypeptides or fragments that specifically bind to the antigen. Examples of antibody fragments include, but are not limited to, a Fab, Fab′, F(ab′)2, Fd, Fv, scFv and scFv-Fc fragment, diabody, triabody, tetrabody, linear antibody, single-chain antibody, and other multispecific antibodies formed from antibody fragments. (See Holliger and Hudson, 2005, Nat. Biotechnol. 23: 1126-1136.) The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. Included in the term immunoglobulin are those immunoglobulin molecules that have modifications in the constant region, including modification (e.g., substitutions, deletions or additions) in amino acid residues that interact with Fey receptors. Antibodies are generally described in, for example, Harlow & Fane, Antibodies: A Faboratory Manual (Cold Spring Harbor Faboratory Press, 1988).

The term “monoclonal antibody” (mAb) refers to an antibody obtained from a population of substantially homogeneous antibodies; that is, the individual antibodies comprising the population are identical except for naturally occurring mutations that may be present in minor amounts. Monoclonal antibodies are highly specific, being directed against a single antigenic determinant, also referred to as an epitope. The modifier “monoclonal” is indicative of a substantially homogeneous population of antibodies directed to the identical epitope and is not to be construed as requiring production of the antibody by any particular method. Monoclonal antibodies can be made by any technique or methodology known in the art; for example, the hybridoma method first described by Kohler et al., 1975, Nature 256:495, or recombinant DNA methods known in the art (see, e.g., U.S. Pat. No. 4,816,567). In another example, monoclonal antibodies can also be isolated from phage antibody libraries, using techniques described in Clackson et al., 1991, Nature 352: 624-628, and Marks et al., 1991, J. Mol. Biol. 222:581-597. In contrast, the antibodies in a preparation of polyclonal antibodies are typically a heterogeneous population of immunoglobulin isotypes and/or classes and also exhibit a variety of epitope specificity.

The term “biosimilar” or “follow-on biologic” or “subsequent entry biologic” refers to a biologic medical product which is almost an identical copy of an original product that is manufactured by a different company. Biosimilars are officially approved versions of original “innovator” products, and can be manufactured when the original product's patent expires. Reference to the innovator product is an integral component of the approval. A biosimilar biological product is highly similar to the reference product notwithstanding minor differences in clinically inactive components, and there are no clinically meaningful differences between the biological product and the reference product in terms of the safety, purity, and potency of the product.

The term “variant” when referring to an antibody as disclosed herein can include any antibody that retains at least some of the activity, e.g., antigen-binding activity, of the reference antibody, but which is structurally different. Variants include fragments of antibodies (e.g., Fab, Fab′ and F(ab′)2, Fd, Fvs, single-chain Fvs (scFv), single-chain antibodies, disulfide-linked Fvs (sdFv) fragments) and also antibodies with altered amino acid sequences, e.g., in the variable domains, due to amino acid substitutions, deletions, or insertions. Variants can occur spontaneously or be intentionally constructed. Intentionally constructed variants can be produced using art-known mutagenesis techniques. Variant antibodies can comprise conservative or non-conservative amino acid substitutions, deletions or additions. The variations are limited by the constraint that the antibody maintains a function of the reference antibody, e.g., binding to the same epitope as the reference antibody, or competitively inhibiting the reference antibody.

The terms “synergy,” “synergism,” or “synergistic” as used herein refer to a combination of therapies (e.g., use of a PI3K inhibitor of Formula (I) and an anti-CD20 antibody) that is more effective than the expected additive effects of any two or more single therapies. For example, a synergistic effect of a combination of therapies permits the use of lower dosages of one or more of the therapies and/or less frequent administration of said therapies to a subject. The ability to utilize lower dosages of therapies and/or to administer the therapies less frequently reduces the toxicity associated with the administration of the therapies to a subject without reducing the efficacy of said therapies in the prevention, management, treatment, or amelioration of a given disease, such as a B cell malignancy. In addition, a synergistic effect can result in improved efficacy of therapies in the prevention, management, treatment, or amelioration of a given disease, such as a B cell malignancy. Finally, synergistic effects of a combination of therapies may avoid or reduce adverse or unwanted side effects associated with the use of any single therapy. The “synergy,” “synergism,” or “synergistic” effect of a combination may be determined herein by the methods of Chou et al., and/or Clarke et al. See Ting-Chao Chou, Theoretical Basis, Experimental Design, and Computerized Simulation of Synergism and Antagonism in Drug Combination Studies, Pharmacol Rev 58:621-681 (2006), and Clarke et al., Issues in experimental design and endpoint analysis in the study of experimental cytotoxic agents in vivo in breast cancer and other models, Breast Cancer Research and Treatment 46:255-278 (1997), which are both incorporated by reference for the methods of determining the “synergy,” synergism,” or “synergistic” effect of a combination.

The term “isotopic variant” refers to a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (ⁿC), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵C1), chlorine-36 (³⁶C1), chlorine-37 (³⁷C1), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵C1), chlorine-37 (³⁷C1), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (H), carbon-11 (C), carbon-14 (C), nitrogen-13 (N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶C1), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a compound contains unnatural proportions of deuterium (D).

The term “alkyl” refers to a linear or branched saturated monovalent hydrocarbon radical, wherein the alkylene may optionally be substituted with one or more substituents Q as described herein. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

The term “alkylene” refers to a linear or branched saturated divalent hydrocarbon radical, wherein the alkylene may optionally be substituted with one or more substituents Q as described herein. The term “alkylene” encompasses both linear and branched alkylene, unless otherwise specified. In certain embodiments, the alkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene, isobutylene, t-butylene, pentylene (including all isomeric forms), and hexylene (including all isomeric forms). For example, C₁₋₆ alkylene refers to a linear saturated divalent hydrocarbon radical of 1 to 6 carbon atoms or a branched saturated divalent hydrocarbon radical of 3 to 6 carbon atoms.

The term “heteroalkylene” refers to a linear or branched saturated divalent hydrocarbon radical that contains one or more heteroatoms each independently selected from O, S, and N in the hydrocarbon chain. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —CH₂OCH₂—, —CH₂CH₂O—, —CH₂NH—, —CH₂NHCH₂—, —CH₂CH₂NH—, —CH₂S—, —CH₂SCH₂—, and —CH₂CH₂S—. In certain embodiments, heteroalkylene may also be optionally substituted with one or more substituents Q as described herein.

The term “alkenyl” refers to a linear or branched monovalent hydrocarbon radical, which contains one or more, in one embodiment, one, two, three, four, or five, in another embodiment, one, carbon-carbon double bond(s). The alkenyl may be optionally substituted with one or more substituents Q as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkenylene” refers to a linear or branched divalent hydrocarbon radical, which contains one or more, in one embodiment, one, two, three, four, or five, in another embodiment, one, carbon-carbon double bond(s). The alkenylene may be optionally substituted with one or more substituents Q as described herein. Similarly, the term “alkenylene” also embraces radicals having “cis” and “trans” configurations, or alternatively, “E” and “Z” configurations. As used herein, the term “alkenylene” encompasses both linear and branched alkenylene, unless otherwise specified. For example, C₂₋₆ alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenylene groups include, but are not limited to, ethenylene, allylene, propenylene, butenylene, and 4-methylbutenylene.

The term “heteroalkenylene” refers to a linear or branched divalent hydrocarbon radical, which contains one or more, in one embodiment, one, two, three, four, or five, in another embodiment, one, carbon-carbon double bond(s), and which contains one or more heteroatoms each independently selected from O, S, and N in the hydrocarbon chain. The heteroalkenylene may be optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, four, or five, in another embodiment, one, carbon-carbon triple bond(s). The alkynyl may be optionally substituted with one or more substituents Q as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “cycloalkyl” refers to a cyclic saturated bridged and/or non-bridged monovalent hydrocarbon radical, which may be optionally substituted with one or more substituents Q as described herein. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, bicyclo[2.1.1]hexyl, bicyclo[2.2.1]heptyl, decalinyl, and adamantyl.

The term “cycloalkenyl” refers to a cyclic unsaturated, nonaromatic bridged and/or non-bridged monovalent hydrocarbon radical, which may be optionally substituted with one or more substituents Q as described herein. In certain embodiments, the cycloalkenyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclobutenyl, cyclopentenyl, cyclohexenyl, or cycloheptenyl,

The term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may be optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, 2-phenylethyl, and 3-phenylpropyl. In certain embodiments, the aralkyl are optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms independently selected from O, S, N, and P in the ring. A heteroaryl group is bonded to the rest of a molecule through its aromatic ring. Each ring of a heteroaryl group can contain one or two O atoms, one or two S atoms, one to four N atoms, and/or one or two P atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, the heteroaryl may also be optionally substituted with one or more substituents Q as described herein as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms independently selected from O, S, N, and P; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. A heterocyclyl group is bonded to the rest of a molecule through its non-aromatic ring. In certain embodiments, the heterocyclyl is a monocyclic, bicyclic, tricyclic, or tetracyclic ring system, which may be spiro, fused, or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindobnyl, isothiazobdinyl, isoxazolidinyl, morpholinyl, octahydroindolyl, octahydroisoindolyl, oxazolidinonyl, oxazobdinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinobnyl, and 1,3,5-trithianyl. In certain embodiments, the heterocyclyl may also be optionally substituted with one or more substituents Q as described herein.

The terms “halogen,” “halide,” or “halo” refer to fluorine, chlorine, bromine, and/or iodine.

The term “optionally substituted” is intended to mean that a group or substituent, such as an alkyl, alkylene, heteroalkylene, alkenyl, alkenylene, heteroalkenylene, alkynyl, cycloalkyl, cycloalkenyl, aryl, aralkyl, heteroaryl, heteroaryl-C₁₋₆ alkyl, and heterocyclyl group, may be substituted with one or more substituents Q, each of which is independently selected from, e.g., (a) oxo (═O), halo, cyano (—CN), and nitro (—NO₂); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, four, or five, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)R^{a}R^{d}, —P(O)(OR^{a})R^{d}, —P(O)(OR^{a})(OR^{d}), —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heteroaryl or heterocyclyl, optionally substituted with one or more, in one embodiment, one, two, three, or four, substituents Q^{a}. As used herein, all groups that can be substituted are “optionally substituted,” unless otherwise specified.

In one embodiment, each substituent Q^{a} is independently selected from the group consisting of (a) oxo, cyano, halo, and nitro; and (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —P(O)R^{e}R^{h}, —P(O)(OR^{e})R^{h}, —P(O)(OR^{e})(OR^{h}), —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) R^{f} and R^{g} together with the N atom to which they are attached form heteroaryl or heterocyclyl.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, Rand S.

The phrase “an enantiomer, a mixture of enantiomers, a mixture of two or more diastereomers, or an isotopic variant thereof; or a pharmaceutically acceptable salt, solvate, hydrate, or prodrug thereof has the same meaning as the phrase “an enantiomer, a mixture of enantiomers, a mixture of two or more diastereomers, or an isotopic variant of the compound referenced therein; or a pharmaceutically acceptable salt, solvate, hydrate, or prodrug of the compound referenced therein; or a pharmaceutically acceptable salt, solvate, hydrate, or prodrug of an enantiomer, a mixture of enantiomers, a mixture of two or more diastereomers, or an isotopic variant of the compound referenced therein.”

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The terms “resistant,” “relapsed,” or “refractory” refer to a cancer that has a reduced responsiveness to a treatment, e.g., the point at which the cancer does not respond to attempted forms of treatment. The cancer can be resistant at the beginning of treatment or it may become resistant during treatment. The term “refractory” can refer to a cancer for which treatment (e.g., chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

The terms “intermittent dosing schedule” or “IS” refer to drugs (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) dosed or administered less than once daily. In some embodiments herein, IS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof), to a subject once daily for a period of about 7 days in a 28-day cycle. In other embodiments herein, IS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) daily for up to three (e.g., two) 28-day cycles and, in the third cycle and subsequent cycles, dosing or administration of the drug to the subject once daily for a period of about 7 days in a 28-day cycle. In some embodiments, IS is continued until progression of disease occurs/is observed or until an incidence of at least one toxicity is reduced.

The terms “continuous dosing schedule” or “CS” refer to drugs (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) dosed or administered once daily. In some embodiments herein, CS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof), to a subject daily in a 28-day cycle. In other embodiments herein, CS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) daily for >three 28-day cycles and, in the one or more subsequent cycles, the drug is dosed or administered to the subject once daily for a period of about 7 days in a 28-day cycle (i.e., late switch to IS). In some embodiments, the subject on CS is never switched to IS. In some embodiments, CS is continued until intolerable toxicity occurs/is observed.

“Responsiveness” or to “respond” to treatment, and other forms of this term, as used herein, refer to the reaction of a subject to treatment with a therapeutic, e.g., a PI3K inhibitor, alone or in combination, e.g., monotherapy or combination therapy. Responsiveness to a therapy, e.g., treatment with a PI3K inhibitor alone or in combination, can be evaluated by comparing a subject's response to the therapy using one or more clinical criteria, such as IWCLL 2008 (for CLL) described in, e.g., Hallek, M. et al. (2008) Blood 111 (12): 5446-5456; the Lugano Classification described in, e.g., Cheson, B. D. et al. Journal of Clinical Oncology, 32(27): 3059-3067; and the like. Additional classifications of responsiveness are provided by. These criteria provide a set of published rules that define when cancer patients improve (“respond”), stay the same (“stable”) or worsen (“progression”) during treatments.

For example, a subject having CLL can be determined to be in complete remission (CR) or partial remission (PR). For example, according to IWCLL 2008, a subject is considered to be in CR if at least all of the following criteria as assessed after completion of therapy are met: (i) Peripheral blood lymphocytes (evaluated by blood and different count) below 4×10⁹/L (4000 μi); (ii) no hepatomegaly or splenomegaly by physical examination; (iii) absence of constitutional symptoms; and (iv) blood counts (e.g., neutrophils, platelets, hemoglobin) above the values set forth in Hallek, M. et al. Partial remission (PR) for CLL is defined according to IWCLL 2008 as including one of: (i) a decrease in number of blood lymphocytes by 50% or more from the value before therapy; (ii) a reduction in lymphadenopathy, as detected by CT scan or palpation; or (iii) a reduction in pretreatment enlargement of spleen or liver by 50% or more, as detected by CT scan or palpation; and blood counts (e.g., neutrophils, platelets, hemoglobin) according to the values set forth in Hallek, M. et al. In other embodiments, a subject having CLL is determined to have progressive disease (PD) or stable disease (SD). For example, according to IWCLL 2008, a subject is considered to be in PD during therapy or after therapy if at least one of the following criteria is met: (i) progression on lymphadenopathy; (ii) an increase in pretreatment enlargement of spleen or liver by 50% or more, or de novo appearance of hepatomegaly or splenomegaly; (iii) an increase in the number of blood lymphocytes by 50% or more with at least 5000 B lymphocytes per microliter; (iv) transformation to a more aggressive histology (e.g., Richter syndrome); or (v) occurrence of cytopenia (neutropenia, anemia or thrombocytopenia) attributable to CLL. Stable disease (SD) for CLL is defined according to IWCLL 2008 as a patient who has not achieved CR or a PR, and who has not exhibited progressive disease.

For example, in some embodiments, a subject with CLL responds to treatment with a PI3K inhibitor, alone or in combination, if at least one of the criteria for disease progression according to IWCLL is retarded or reduced, e.g., by about 10%, 20%, 30%, 40%, 50%, 60%, 70%, 80%, 90% or more. In another example, a subject responds to treatment with a PI3K inhibitor, alone or in combination, if the subject experiences a life expectancy extension, e.g., extended by about 5%, 10%, 20%, 30%, 40%, 50% or more beyond the life expectancy predicted if no treatment is administered. In another example, a subject responds to treatment with a PI3K inhibitor, alone or in combination, if the subject has one or more of: an increased progression-free survival, overall survival or increased time to progression (TTP), e.g., as described in Hallek, M. et al.

### PI3K Inhibitors

Some embodiments provided herein describe PI3K inhibitors useful for treating relapsed cancer patients identified as early progressors. In some embodiments, the PI3K inhibitor is selective for PI3K delta. Provided here in some embodiments are PI3K inhibitors of Formula (I):

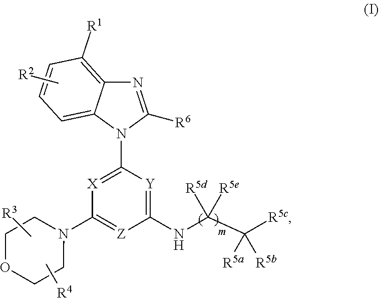

or an enantiomer, a mixture of enantiomers, a mixture of two or more diastereomers, or an isotopic variant thereof; or a pharmaceutically acceptable salt, solvate, hydrate, or prodrug thereof; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, four, or five substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein two substituents Q that are adjacent to each other optionally form a C₃₋₁₀ cycloalkenyl, C₆₋₁₄ aryl, heteroaryl, or heterocyclyl, each optionally substituted with one, two, three, or four substituents Q^{a}.

In some embodiments, the compound of structural Formula (I) is not 4-(2-(difluoromethyl)-1H-benzo[r/]imidazol-1-yl)-6-morpholino-/V-(2-phenyl-2-(pyrrolidin-1-yl)ethyl)-1,3,5-triazin-2-amine or 6-(2-(difluoromethyl)-1H-benzo[d]imidazol-1-yl)-N-(1-(4-((R)-3-(methoxymethyl)morpholino)phenyl)ethyl)-2-morpholinopyrimidin-4-amine.

In one embodiment of a compound of Formula (I), X, Y, and Z are each independently N or CR^{X} with the proviso that at least two of X, Y, and Z are nitrogen atoms; where R^{X} is hydrogen or C₁₋₆ alkyl. In another embodiment of a compound of Formula (I), X, Y, and Z are N. In some embodiments, R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments, R^{5a} and R^{5b} are each independently
(a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a},
—S(O)NR^{1b}R^{1c}, or —S(O)₂NR^{1b}R^{1c}.

In some embodiments, R^{5a} and R^{5b} are each methyl, optionally substituted with one or more halo.

In some embodiments, R^{5f} and R^{5g} are each hydrogen.

In some embodiments of compounds of structural Formula (I):
X, Y, and Z are each N;
R¹ and R² are each hydrogen;
R³ and R⁴ are each hydrogen;
R^{5a} is C₁₋₆ alkyl;
R^{5b} is C_{w} alkyl;
R^{5c} is —(CH₂)-phenyl, wherein R^{5c} is optionally substituted with one, two, three, or four substituents Q;
R^{5d} and R^{5e} are each hydrogen;
R⁶ is CHF₂; and
m is 0;
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

In some embodiments of compounds of structural Formula (I):
X, Y, and Z are each N;
R¹ and R² are each hydrogen;
R³ and R⁴ are each hydrogen;
R^{5a} and R^{5b} are each methyl, optionally substituted with one or more halo;
R^{5c} is —(CH₂)-phenyl, wherein R^{5c} is optionally substituted with one, two, three, or four
substituents Q;
R^{5d} and R^{5e} are each hydrogen;
R⁶ is CHF₂; and
m is 0;
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

Provided herein is a compound of Formula (II):

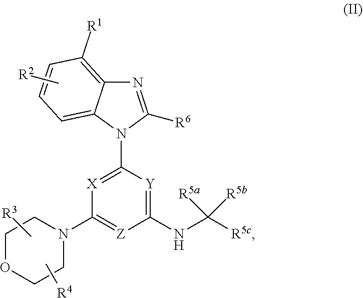

or an enantiomer, a mixture of enantiomers, a mixture of two or more diastereomers, or an isotopic variant thereof; or a pharmaceutically acceptable salt, solvate, hydrate, or prodrug thereof. In some embodiments, R^{5c} is C₆₋₁₄ aryl, optionally substituted with one or more substituents Q. In some embodiments, R^{5c} is phenyl, optionally substituted with one or more substituents Q. In some embodiments, R^{5c} is naphthyl, optionally substituted with one or more substituents Q. In some embodiments, R^{5c} is —(CR^{5f}R^{5g})ₙ—(C₆₋₁₄ aryl), wherein the aryl is optionally substituted with one or more substituents Q. In some embodiments, R^{5c} is —(CH₂)-phenyl, wherein the phenyl is optionally substituted with one or more substituents Q. In some embodiments, R^{5c} is —(CH₂)-naphthyl, wherein the naphthyl is optionally substituted with one or more substituents Q. In some embodiments, R^{5c} is heteroaryl, optionally substituted with one or more substituents Q. In some embodiments, R^{5c} is monocyclic heteroaryl, optionally substituted with one or more substituents Q. In some embodiments, R^{5c} is 5- or 6-membered heteroaryl, optionally substituted with one or more substituents Q. In some embodiments, R^{5c} is bicyclic heteroaryl, optionally substituted with one or more substituents Q. In some embodiments, R^{5c} is —(CR^{5f}R^{5g})ₙ-heteroaryl, wherein the heteroaryl is optionally substituted with one or more substituents Q. In some embodiments, R^{5c} is —(CR^{5f}R^{5g})ₙ-(monocyclic heteroaryl), wherein the heteroaryl is optionally substituted with one or more substituents Q. R^{5c} is —(CR^{5f}R^{5g})ₙ-(5- or 6-membered heteroaryl), wherein the heteroaryl is optionally substituted with one or more substituents Q. In some embodiments, R^{5c} is —(CR^{5f}R^{5g})ₙ-(bicyclic heteroaryl), wherein the heteroaryl is optionally substituted with one or more substituents Q.

Also provided herein is a compound of Formula (VII):

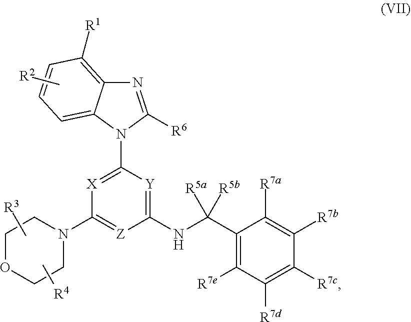

or an enantiomer, a mixture of enantiomers, a mixture of two or more diastereomers, or an isotopic variant thereof; or a pharmaceutically acceptable salt, solvate, hydrate, or prodrug thereof, wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one or more substituents Q.

Also provided herein is a compound of Formula (IX):

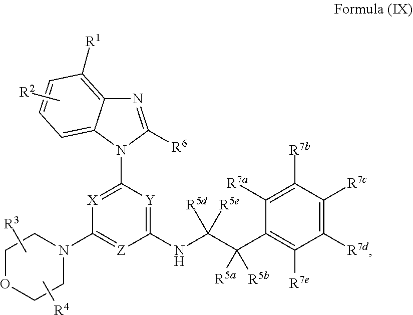

or an enantiomer, a mixture of enantiomers, a mixture of two or more diastereomers, or an isotopic variant thereof; or a pharmaceutically acceptable salt, solvate, hydrate, or prodrug thereof, wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

In some embodiments, R^{7a} is hydrogen, halo, C₁₋₆ alkyl optionally substituted with one or more substituents Q, or —OR^{1a}.

In some embodiments, R^{7a} is hydrogen. In some embodiments, R^{7a} is (a) cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}. In some embodiments, R^{a} is (i) halo; (ii) C₁₋₆ alkyl, C₆₋₁₄ aryl, C₇₋₁₅ aralkyl, heteroaryl, or heterocyclyl, each of which is optionally substituted with one or more substituents Q; or (iii) —OR^{1a} or —NR^{1b}R^{1c}.

In some embodiments, R^{7b} is hydrogen, halo, C₁₋₆ alkyl optionally substituted with one or more substituents Q, or —OR^{1a}. In some embodiments, R^{7b} is hydrogen.

In some embodiments, R^{7c} is hydrogen, halo, C₁₋₆ alkyl optionally substituted with one or more substituents Q, or —OR^{1a}. In some embodiments, R^{7c} is hydrogen, halo, or —OR^{1a}. In some embodiments, R^{7C} is chloro. In some embodiments, R^{7c} is —O—C₁₋₆ alkyl, optionally substituted with one or more substituents Q.

In some embodiments, R^{7d} is hydrogen, halo, C₁₋₆ alkyl optionally substituted with one or more substituents Q, or —OR^{1a}. In some embodiments, R^{7d} is hydrogen.

In some embodiments, R^{7e} is hydrogen, halo, C₁₋₆ alkyl optionally substituted with one or more substituents Q, or —OR^{1a}. In some embodiments, R^{7e} is hydrogen. In some embodiments, two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one or more substituents Q. In some embodiments, R^{7a} and R^{7b} together with the carbon atoms to which they are attached form C₆₋₁₄ aryl, optionally substituted with one or more substituents Q.

In some embodiments, R^{5a} is hydrogen. In some embodiments, R^{5a} is C₁₋₆ alkyl, optionally substituted with one or more substituents Q. In some embodiments, R^{5a} is hydrogen, methyl, or ethyl.

In some embodiments, R^{5b} is C₁₋₆ alkyl, optionally substituted with one or more substituents Q. In some embodiments, R^{5b} is methyl, ethyl, or propyl. In some embodiments, R^{5b} is —C(O)OR^{1a}. In some embodiments, R^{5b} is —C(O)O—C₁₋₆ alkyl. In some embodiments, R^{5b} is —C(O)OCH₃.

Also provided herein is a compound of Formula (X):

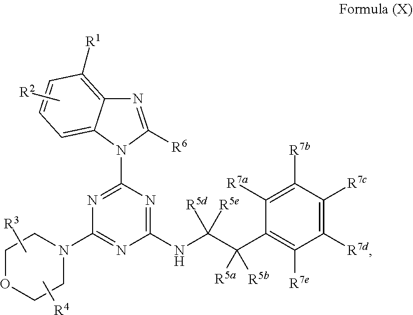

or an enantiomer, a mixture of enantiomers, a mixture of two or more diastereomers, or an isotopic variant thereof; or a pharmaceutically acceptable salt, solvate, hydrate, or prodrug thereof.

Provided herein is a compound of Formula (XI):

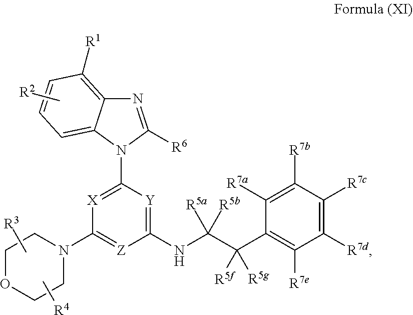

or an enantiomer, a mixture of enantiomers, a mixture of two or more diastereomers, or an isotopic variant thereof; or a pharmaceutically acceptable salt, solvate, hydrate, or prodrug thereof,
wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

In certain embodiments, R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}. In certain embodiments, one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; in certain embodiments, one of R^{7a}, R^{7b}, R^{7c}, R^{7d}, and R^{7e} is C₆₋₁₄ aryl, e.g., phenyl, optionally substituted with one, two, three, or four substituents Q^{a}; in certain embodiments, one of R^{7a}, R^{7b}, R^{7c}, R^{7d}, and R^{7e} is heteroaryl, e.g., 5-membered or 6-membered heteroaryl, optionally substituted with one, two, three, or four substituents Q^{a}; in certain embodiments, one of R^{7a}, R^{7b}, R^{7c}, R^{7d}, and R^{7e} is heterocyclyl, e.g., 5-membered or 6-membered heterocyclyl, optionally substituted with one, two, three, or four substituents Q^{a}; in certain embodiments, one of R^{7a}, R^{7b}, R^{7c}, R^{7d}, and R^{7e} is phenyl, imidazolyl, pyrozolyl, pyridinyl, piperidinyl, or piperazinyl, each optionally substituted with one, two, three, or four substituents Q^{a}; in certain embodiments, one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one or more substituents Q^{a}; in certain embodiments, one of R^{7a}, R^{7b}, R^{7c}, R^{7d}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, l-methylpiperidin-4-yl, or 4-methylpiperazin-1-yl; and in certain embodiments, one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, l-ethylpiperidin-4-yl, 1-isopropylpiperidin-4-yl, l-acetylpiperidin-4-yl, l-methylsulfonylpiperidin-4-yl, or 4-methylpiperazin-1-yl.

In certain embodiments, R^{7a} is C₆₋₁₄ aryl, heteroaryl, or heterocyclyl, each of which is optionally substituted with one, two, three, or four substituents Q^{a}; in certain embodiments, R^{7a} is C₆₋₁₄ aryl, e.g., phenyl, optionally substituted with one, two, three, or four substituents Q^{a}; in certain embodiments, R^{7a} is heteroaryl, e.g., 5-membered or 6-membered heteroaryl, optionally substituted with one, two, three, or four substituents Q^{a}; in certain embodiments, R^{7a} is heterocyclyl, e.g., 5-membered or 6-membered heterocyclyl, optionally substituted with one, two, three, or four substituents Q^{a}; in certain embodiments, R^{a} is phenyl, imidazolyl, pyrozolyl, pyridinyl, piperidinyl, or piperazinyl, each optionally substituted with one, two, three, or four substituents Q^{a}; in certain embodiments, R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; in certain embodiments, R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, l-methylpiperidin-4-yl, or 4-methylpiperazin-1-yl; and in certain embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, l-methylpyrrolidin-3-yl, piperidin-4-yl, 1-methylpiperidin-4-yl, l-ethylpiperidin-4-yl, l-isopropylpiperidin-4-yl, 1-acetylpiperidin-4-yl, 1-methylsulfonylpiperidin-4-yl, or 4-methylpiperazin-1-yl.

In certain embodiments:

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently C₁₋₆ alkyl, optionally substituted with one, two, three, four, or five substituents Q;

R^{5f} and R^{5g} are each independently hydrogen, halo, C₁₋₆ alkyl, optionally substituted with one, two, three, four, or five substituents Q; or R^{5f} and R^{5g} together with the carbon atom to which they are attached form C₁₋₁₀ cycloalkyl or heterocyclyl, each of which is optionally substituted with one, two, three, four, or five substituents Q;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CR^{X}, with the proviso that at least two of X, Y, and Z are N; where R^{x} is a hydrogen or C₁₋₆ alkyl, optionally substituted with one, two, three, or four substituents Q^{a}.

In certain embodiments:
R¹ is hydrogen or methoxy;
R² is hydrogen;
R³ and R⁴ are hydrogen;
R⁶ is C₁₋₆ alkyl, optionally substituted with one or more halo;
R^{5a} and R^{5b} are each independently C₁₋₆ alkyl;
R^{5f} and R^{5g} are each independently hydrogen or C₁₋₆ alkyl; or R^{5f} and R^{5g} together with the carbon atom to which they are attached form C₁₋₁₀ cycloalkyl;
R^{a} is C₆₋₁₄ aryl, heteroaryl, or heterocyclyl, each of which is optionally substituted with one, two, three, or four substituents Q^{a};
R^{7b}, R^{7c}, R^{7d}, and R^{7e} are hydrogen; and
X, Y, and Z are each independently N or CH.

In certain embodiments:
R¹ is hydrogen or methoxy;
R² is hydrogen;
R³ and R⁴ are hydrogen;
R⁶ is difluoromethyl;
R^{5a} and R^{5b} are methyl;
R^{5f} and R^{5g} are hydrogen; or R^{5f} and R^{5g} together with the carbon atom to which they are attached form cyclopropyl, cyclobutyl, cyclopentyl, or cyclohexyl;
R^{7a} is C₆₋₁₄ aryl, monocyclic heteroaryl, or monocyclic heterocyclyl, each of which is optionally substituted with one, two, three, or four substituents Q^{a};
R^{7b}, R^{7c}, R^{7d}, and R^{7e} are hydrogen; and
X, Y, and Z are each independently N or CH.

In certain embodiments:
R¹ is hydrogen or methoxy;
R² is hydrogen;
R³ and R⁴ are hydrogen;
R⁶ is difluoromethyl;
R^{5a} and R^{5b} are methyl;
R^{5f} and R^{5g} are hydrogen; or R^{5f} and R^{5g} together with the carbon atom to which they are attached form cyclopropyl, cyclobutyl, cyclopentyl, or cyclohexyl;
R^{7a} is phenyl, 5- or 6-membered heteroaryl, or 5- or 6-membered heterocyclyl, each of which is optionally substituted with one, two, three, or four substituents Q^{a};
R^{7b}, R^{7c}, R^{7d}, and R^{7e} are hydrogen; and
X, Y, and Z are each independently N or CH.

In certain embodiments:
R¹ is hydrogen or methoxy;
R² is hydrogen;
R³ and R⁴ are hydrogen;
R⁶ is difluoromethyl;
R^{5a} and R^{5b} are methyl;
R^{5f} and R^{5g} are hydrogen; or R^{5f} and R^{5g} together with the carbon atom to which they are attached form cyclopropyl, cyclobutyl, cyclopentyl, or cyclohexyl;
R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a};
R^{7b}, R^{7c}, R^{7d}, and R^{7e} are hydrogen; and
X, Y, and Z are each independently N or CH.

In certain embodiments, R^{7a} is phenyl, imidazolyl, pyrozolyl, pyridinyl, piperidinyl, or piperazinyl, each of which is optionally substituted with one, two, three, or four substituents Q^{a}.

Provided herein is a compound of Formula (XVI):

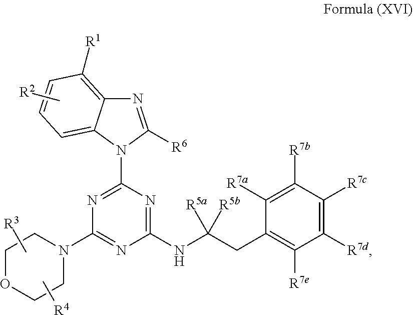

or an enantiomer, a mixture of enantiomers, a mixture of two or more diastereomers, or an isotopic variant thereof; or a pharmaceutically acceptable salt, solvate, hydrate, or prodrug thereof.

In some embodiments, R^{5a} is C₁₋₆ alkyl, optionally substituted with one or more substituents Q. In some embodiments, R^{5a} is methyl.

In some embodiments, R^{5b} is C₁₋₆ alkyl, optionally substituted with one or more substituents Q. In some embodiments, R^{5b} is methyl.

In some embodiments, R^{5a} and R^{5b} are methyl.

In some embodiments, R^{7a} is hydrogen, halo, C₁₋₆ alkyl, C₆₋₁₄ aryl, heteroaryl, or heterocyclyl, where the alkyl, aryl, heteroaryl, and heterocyclyl are each optionally substituted with one or more substituents Q. In some embodiments, R^{7a} is C₆₋₁₄ aryl, optionally substituted with one or more substituents Q. In some embodiments, R^{7a} is phenyl, optionally substituted with one or more substituents Q In some embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-florophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, or 3-morpholin-4-ylmethylphenyl. In some embodiments, R^{7a} is heteroaryl, optionally substituted with one or more substituents Q. In some embodiments, R^{7a} is monocyclic heteroaryl, optionally substituted with one or more substituents Q. In some embodiments, R^{7a} is 5- or 6-membered heteroaryl, each optionally substituted with one or more substituents Q. In some embodiments, R^{7a} is imidazolyl, pyrozolyl, pyridinyl, or pyrimidinyl, each optionally substituted with one or more substituents Q. In some embodiments, R^{7a} is imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl. In some embodiments, R^{7a} is heterocyclyl, optionally substituted with one or more substituents Q. In some embodiments, R^{7a} is monocyclic heterocyclyl, optionally substituted with one or more substituents Q. In some embodiments, R^{7a} is 5- or 6-membered heterocyclyl, each optionally substituted with one or more substituents Q. In some embodiments, R^{7a} is pyrrolidinyl, piperidinyl, or piperazinyl, each optionally substituted with one or more substituents Q. In some embodiments, R^{7a} is pyrrolidin-3-yl, l-methylpyrrolidin-3-yl, piperidin-4-yl, 1-methylpiperidin-4-yl, l-ethylpiperidin-4-yl, l-isopropylpiperidin-4-yl, 1-acetylpiperidin-4-yl, 1-methylsulfonylpiperidin-4-yl, or 4-methylpiperazin-1-yl.

In some embodiments, R^{7b} is hydrogen, halo, or C₁₋₆ alkyl optionally substituted with one or more substituents Q. In some embodiments, R^{7b} is hydrogen.

In some embodiments, R^{7c} is hydrogen, halo, or C₁₋₆ alkyl optionally substituted with one or more substituents Q. In some embodiments, R^{7c} is hydrogen.

In some embodiments, R^{7d} is hydrogen, halo, or C₁₋₆ alkyl optionally substituted with one or more substituents Q. In some embodiments, R^{7d} is hydrogen.

In some embodiments, R^{7e} is hydrogen, halo, or C₁₋₆ alkyl optionally substituted with one or more substituents Q. In some embodiments, R^{7e} is hydrogen.

In some embodiments, R^{7a} is C₆₋₄ aryl, heteroaryl, or heterocyclyl, each optionally substituted with one or more substituents Q; and R^{7b}, R^{7c}, R^{7d}, and R^{7e} are hydrogen.

In one embodiment of a compound of Formula (XVI), one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In another embodiment of a compound Formula (XVI), one of R^{7a}, R^{7b}, R^{7c}, R^{7d}, and R^{7e} is C₆₋₁₄ aryl, which is optionally substituted with one, two, three, or four substituents Q^{a}; and R¹, R², R³, R⁴, R⁶, R^{a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is heteroaryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formulae (XVI), one of R^{7a}, R^{7b}, R^{7c}, R^{7d}, and R^{7e} is heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is 5-membered or 6-membered heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, l-methylpyrrolidin-3-yl, piperidin-4-yl, 1-methylpiperidin-4-yl, l-ethylpiperidin-4-yl, l-isopropylpiperidin-4-yl, 1-acetylpiperidin-4-yl, 1-methylsulfonylpiperidin-4-yl, or 4-methylpiperazin-1-yl.

In still another embodiment of a compound of Formula (XVI), one of R^{7a}, R^{7b}, R^{7c}, R^{7d}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, l-methylpiperidin-4-yl, or 4-methylpiperazin-1-yl; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVI), R^{7a} is C₆₋₁₄ aryl, heteroaryl, or heterocyclyl, each of which is optionally substituted with one, two, three, or four substituents Q^{a}; and R¹, R², R³, R⁴, R⁶, R^{a}, R^{5b}, R^{7b}, R^{7c}, R^{7d}, R^{7e}, X, Y, and Z are each as defined herein.

In yet another embodiment of a compound of Formula (XVI), R^{7a} is heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is 5-membered or 6-membered heterocyclyl, which is optionally substituted with one, two, three, or four substituents Q^{a}; and R¹, R², R³, R⁴, R⁶, R^{5a}, R^{5b}, R^{b}, R^{7c}, R^{d}, R^{7e}, X, Y, and Z are each as defined herein.

In yet another embodiment of a compound of Formula (XVI), R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, l-methylpyrrolidin-3-yl, piperidin-4-yl, l-methylpiperidin-4-yl, l-ethylpiperidin-4-yl, 1-isopropylpiperidin-4-yl, l-acetylpiperidin-4-yl, 1-methylsulfonylpiperidin-4-yl, or 4-methylpiperazin-1-yl.

In yet another embodiment of a compound of Formula (XVI), R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, l-methylpiperidin-4-yl, or 4-methylpiperazin-1-yl; and R¹, R², R³, R⁴, R⁶, R^{5a}, R^{5b}, R^{b}, R^{7c}, R^{7d}, R^{7e}, X, Y, and Z are each as defined herein.

In one embodiment of a compound of Formula (XVI),
R¹ is hydrogen or —OR^{1a}, where R^{1a} is C₁₋₆ alkyl, optionally substituted with one, two, three, four, or five substituents Q;
R² is hydrogen;
R³ and R⁴ are hydrogen;
R⁶ is C₁₋₆ alkyl, optionally substituted with one, two, three, four, or five substituents Q;
R^{5a} and R^{5b} are each independently C₁₋₆ alkyl, optionally substituted with one, two, three, four, or five substituents Q;
R^{7a} is C₆₋₁₄ aryl, heteroaryl, or heterocyclyl, each of which is optionally substituted with one or more substituents Q^{a}; and
R^{7b}, R^{7c}, R^{7d}, and R^{7e} are hydrogen.

In one embodiment of a compound of Formula (XVI):
R¹ is hydrogen or methoxy;
R² is hydrogen;
R³ and R⁴ are hydrogen;
R⁶ is C₁₋₆ alkyl, optionally substituted with one or more halo;
R^{5a} and R^{5b} are each independently C₁₋₆ alkyl;
R^{7a} is C₆₋₁₄ aryl, heteroaryl, or heterocyclyl, each of which is optionally substituted with one, two, three, or four substituents Q^{a}; and
R^{7b}, R^{7c}, R^{7d}, and R^{7e} are hydrogen.

In one embodiment of a compound of Formula (XVI):
R¹ is hydrogen or methoxy;
R² is hydrogen;
R³ and R⁴ are hydrogen;
R⁶ is difluoromethyl;
R^{5a} and R^{5b} are methyl;
R^{7a} is phenyl, imidazolyl, pyrozolyl, pyridinyl, pyrimidinyl, pyrrolidinyl, piperidinyl, or piperazinyl, each of which is optionally substituted with one, two, three, four, or five substituents Q; and
R^{7b}, R^{7c}, R^{7d}, and R^{7e} are hydrogen.

In one embodiment of a compound of Formula (XVI), R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; and R¹, R², R³, R⁴, R⁶, R^{7a}, R^{7b}, R^{7c}, R^{7d}, R^{7e}, R^{1a}, R^{1b}, R^{1c}, and R^{1d} are defined herein elsewhere.

In one embodiment of any of the formulae provided herein:
R¹ is hydrogen or —OR^{1a}, where R^{1a} is C₁₋₆ alkyl, optionally substituted with one, two, three, four, or five substituents Q;
R² is hydrogen;
R³ and R⁴ are hydrogen;
R⁶ is C₁₋₆ alkyl, optionally substituted with one, two, three, four, or five substituents Q;
R^{5a} and R^{5b} are each independently hydrogen or C₁₋₆ alkyl optionally substituted with one, two, three, four, or five substituents Q;
R^{7a} is C₆₋₁₄ aryl, heteroaryl, or heterocyclyl, each of which is optionally substituted with one, two, three, or four substituents Q^{a};
R^{7b}, R^{7c}, R^{7d}, and R^{7e} are hydrogen; and
X, Y, and Z are each independently N or CR^{X}, with the proviso that at least two of X, Y, and Z are N; where R^{x} is a hydrogen or C₁₋₆ alkyl, optionally substituted with one, two, three, or four substituents Q^{a}.

In one embodiment of any of the formulae provided herein:
R¹ is hydrogen or methoxy;
R² is hydrogen;
R³ and R⁴ are hydrogen;
R⁶ is C₁₋₆ alkyl, optionally substituted with one or more halo;
R^{5a} and R^{5b} are each independently hydrogen or C₁₋₆ alkyl;
R^{7a} is C₆₋₁₄ aryl, heteroaryl, or heterocyclyl, each of which is optionally substituted with one, two, three, or four substituents Q^{a};
R^{7b}, R^{7c}, R^{7d}, and R^{7e} are hydrogen; and
X, Y, and Z are each independently N or CH.

In one embodiment of any of the formulae provided herein:
R¹ is hydrogen or methoxy;
R² is hydrogen;
R³ and R⁴ are hydrogen;
R⁶ is difluoromethyl;
R^{5a} and R^{5b} are each independently hydrogen or C₁₋₆ alkyl;
R^{7a} is C₆₋₁₄ aryl, monocyclic heteroaryl, or monocyclic heterocyclyl, each of which is optionally substituted with one, two, three, or four substituents Q^{a};
R^{7b}, R^{7c}, R^{7d}, and R^{7e} are hydrogen; and
X, Y, and Z are each independently N or CH.

In one embodiment of any of the formulae provided herein:
R¹ is hydrogen or methoxy;
R² is hydrogen;
R³ and R⁴ are hydrogen;
R⁶ is difluoromethyl;
R^{5a} and R^{5b} are each independently hydrogen or C₁₋₆ alkyl;
R^{7a} is phenyl, 5- or 6-membered heteroaryl, or 5- or 6-membered heterocyclyl, each of which is optionally substituted with one, two, three, or four substituents Q^{a};
R^{7b}, R^{7c}, R^{7d}, and R^{7e} are hydrogen; and
X, Y, and Z are each independently N or CH.

In one embodiment of any of the formulae provided herein:
R¹ is hydrogen or methoxy;
R² is hydrogen;
R³ and R⁴ are hydrogen;
R⁶ is difluoromethyl;
R^{5a} and R^{5b} are each independently hydrogen or C₁₋₆ alkyl;
R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a};
R^{7b}, R^{7c}, R^{7d}, and R^{7e} are hydrogen; and
X, Y, and Z are each independently N or CH.

In one embodiment of any of the formulae provided herein:
R¹ is hydrogen or methoxy;
R² is hydrogen;
R³ and R⁴ are hydrogen;
R⁶ is difluoromethyl;
R^{5a} and R^{5b} are each independently hydrogen or C₁₋₆ alkyl;
R^{7a} is phenyl, imidazolyl, pyrozolyl, pyridinyl, piperidinyl, or piperazinyl, each of which is optionally substituted with one, two, three, or four substituents Q^{a};
R^{7b}, R^{7c}, R^{7d}, and R^{7e} are hydrogen; and
X, Y, and Z are each independently N or CH.

In one embodiment of any of the formulae provided herein, R¹ is hydrogen. In one embodiment of any of the formulae provided herein, R¹ is —OR^{1a}. In one embodiment of any of the formulae provided herein, R¹ is —O—C₁₋₆ alkyl. In one embodiment of any of the formulae provided herein, R¹ is methoxy.

In one embodiment of any of the formulae provided herein, R² is hydrogen. In one embodiment of any of the formulae provided herein, R² is —NR^{1b}R^{1c}. In one embodiment of any of the formulae provided herein, R² is amino.

In one embodiment of any of the formulae provided herein, R³ is hydrogen.

In one embodiment of any of the formulae provided herein, R⁴ is hydrogen.

In one embodiment of any of the formulae provided herein, R⁶ is C₁₋₆ alkyl, optionally substituted with one or more substituents Q.

In one embodiment of any of the formulae provided herein, R⁶ is methyl, fluoromethyl, difluoromethyl, or trifluoromethyl. In one embodiment of any of the formulae provided herein, R⁶ is difluoromethyl.

The groups or variables, R¹, R², R³, R⁴, R⁶, R^{5a}, R^{5b}, R^{5c}, R^{5d}, R^{5e}, R^{5f}, R^{5g}, R^{7a}, R^{7b}, R^{7c}, R^{7d}, R^{7e}, m, n, X, Y, and Z in Formulae provided herein, e.g., Formulae (I), (II), (VII), (IX), (X), (XI), (XVI), are further defined in the embodiments described herein. All combinations of the embodiments provided herein for such groups and/or variables are within the scope of this disclosure.

In certain embodiments, m is 0. In certain embodiments, m is 1.

In certain embodiments, n is 0. In certain embodiments, n is 1. In certain embodiments, n is 2. In certain embodiments, n is 3. In certain embodiments, n is 4. In certain embodiments, n is 0, 1, or 2. In certain embodiments, n is 0, 1, 2, or 3. In certain embodiments, n is 1, 2, or 3. In certain embodiments, n is 1 or 2.

In certain embodiments, m is 0, and n is 0, 1, 2, or 3. In certain embodiments, m is 0, n is 0, 1, or 2. In certain embodiments, m is 0, n is 0 or 1. In certain embodiments, m is 0, n is 0. In certain embodiments, m is 0 and n is 1. In certain embodiments, m is 1, n is 0, 1, 2, or 3. In certain embodiments, m is 1, n is 0, 1, or 2. In certain embodiments, m is 1, n is 0 or 1. In certain embodiments, m is 1, n is 0. In certain embodiments, m is 1, n is 1.

In specific embodiments, m is 0, n is 1, and R^{5a} and R^{5b} are each methyl.

In certain embodiments, X is N. In certain embodiments, X is CR^{X}, wherein R^{x} is as defined herein. In certain embodiments, X is CH.

In certain embodiments, Y is N. In certain embodiments, Y is CR^{X}, wherein R^{x} is as defined herein. In certain embodiments, Y is CH.

In certain embodiments, Z is N. In certain embodiments, Z is CR^{X}, wherein R^{x} is as defined herein. In certain embodiments, Z is CH.

In certain embodiments, X, Y, and Z are N. In certain embodiments, X and Y are N, and Z is CH.

In certain embodiments, X and Z are N, and Y is CH. In certain embodiments, Y and Z are N, and X is CH.

In certain embodiments, the compound provided herein is not 4-(2-(difluoromethyl)-1H-benzo[d]imidazol-1-yl)-6-morpholino-/V-(2-phenyl-2-(pyrrolidin-1-yl)ethyl)-1,3,5-triazin-2-amine. In certain embodiments, the compound provided herein is not 6-(2-(difluoromethyl)-1H-benzo[d]imidazol-1-yl)-N-(1-(4-((R)-3-(methoxymethyl)morpholino)phenyl)ethyl)-2-morpholinopyrimidin-4-amine.

In certain embodiments, when X, Y, and Z are N, and R^{5a} is hydrogen, R^{5b} is not heterocyclyl. In certain embodiments, when X, Y, and Z are N, and R^{5a} is hydrogen, R^{5b} is not 5-membered heterocyclyl.

In certain embodiments, when X, Y, and Z are N, and R^{5a} is hydrogen, R^{5b} is not pyrrolidinyl. In certain embodiments, when X, Y, and Z are N, and R^{5a} is hydrogen, R^{5b} is not pyrrolidin-1-yl.

In certain embodiments, when X and Z are N, Y is CH, and R^{5a} is hydrogen, R^{5b} is morpholino-substituted phenyl. In certain embodiments, when X and Z are N, Y is CH, and R^{5a} is hydrogen, R^{5b} is not 4-((R)-3-(methoxymethyl)morpholino)phenyl.

In one embodiment, provided herein is a compound selected from:

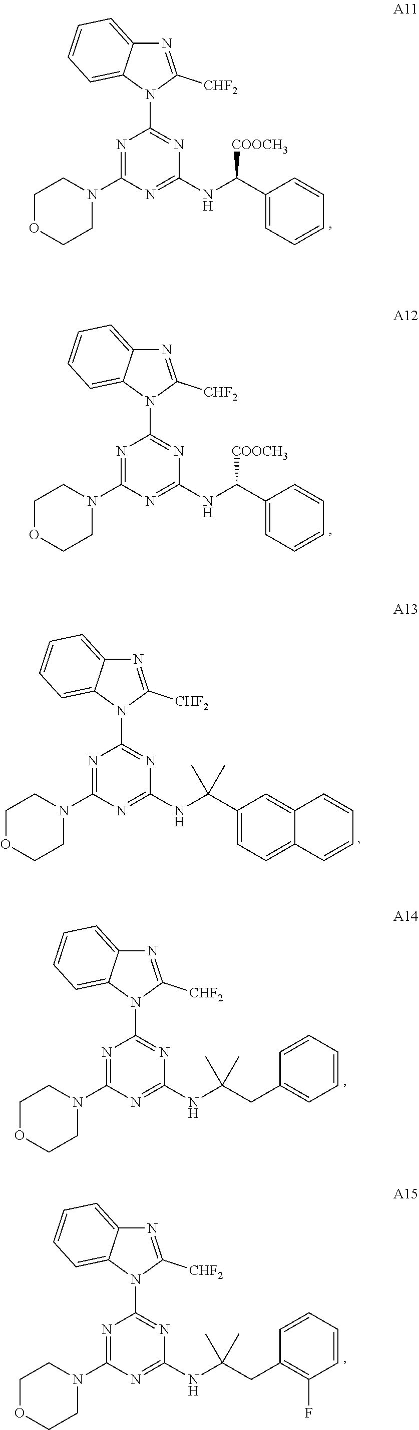 [SMILES: COC(=O)[C@H](Nc1nc(N2CCOCC2)nc(-n2c(C(F)F)nc3ccccc32)n1)c1ccccc1 ; COC(=O)[C@@H](Nc1nc(N2CCOCC2)nc(-n2c(C(F)F)nc3ccccc32)n1)c1ccccc1 ; CC(C)(Nc1nc(N2CCOCC2)nc(-n2c(C(F)F)nc3ccccc32)n1)c1ccc2ccccc2c1 ; CC(C)(Cc1ccccc1)Nc1nc(N2CCOCC2)nc(-n2c(C(F)F)nc3ccccc32)n1 ; CC(C)(Cc1ccccc1F)Nc1nc(N2CCOCC2)nc(-n2c(C(F)F)nc3ccccc32)n1]

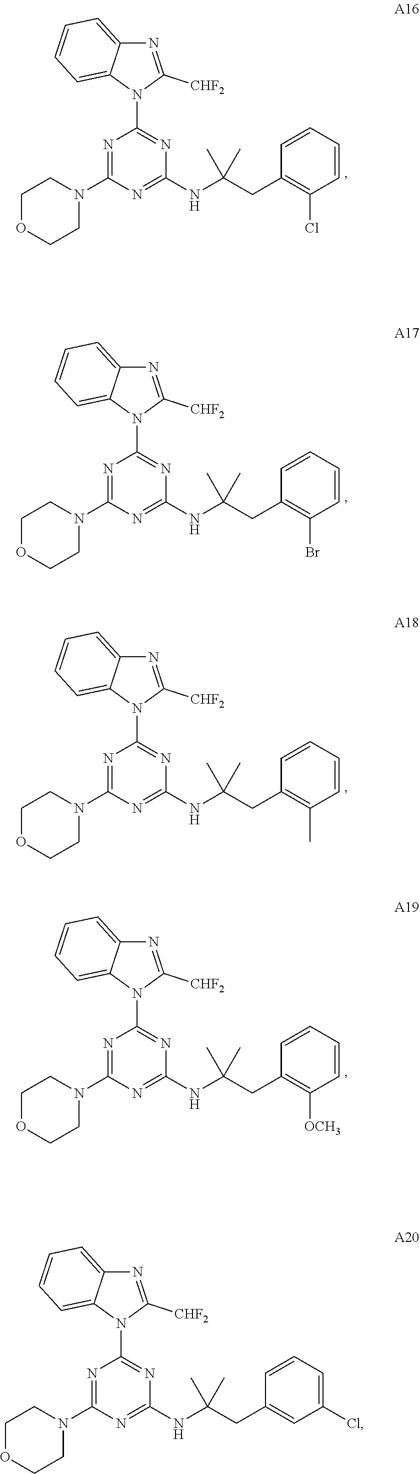 [SMILES: CC(C)(Cc1ccccc1Cl)Nc1nc(N2CCOCC2)nc(-n2c(C(F)F)nc3ccccc32)n1 ; CC(C)(Cc1ccccc1Br)Nc1nc(N2CCOCC2)nc(-n2c(C(F)F)nc3ccccc32)n1 ; Cc1ccccc1CC(C)(C)Nc1nc(N2CCOCC2)nc(-n2c(C(F)F)nc3ccccc32)n1 ; COc1ccccc1CC(C)(C)Nc1nc(N2CCOCC2)nc(-n2c(C(F)F)nc3ccccc32)n1 ; CC(C)(Cc1cccc(Cl)c1)Nc1nc(N2CCOCC2)nc(-n2c(C(F)F)nc3ccccc32)n1]

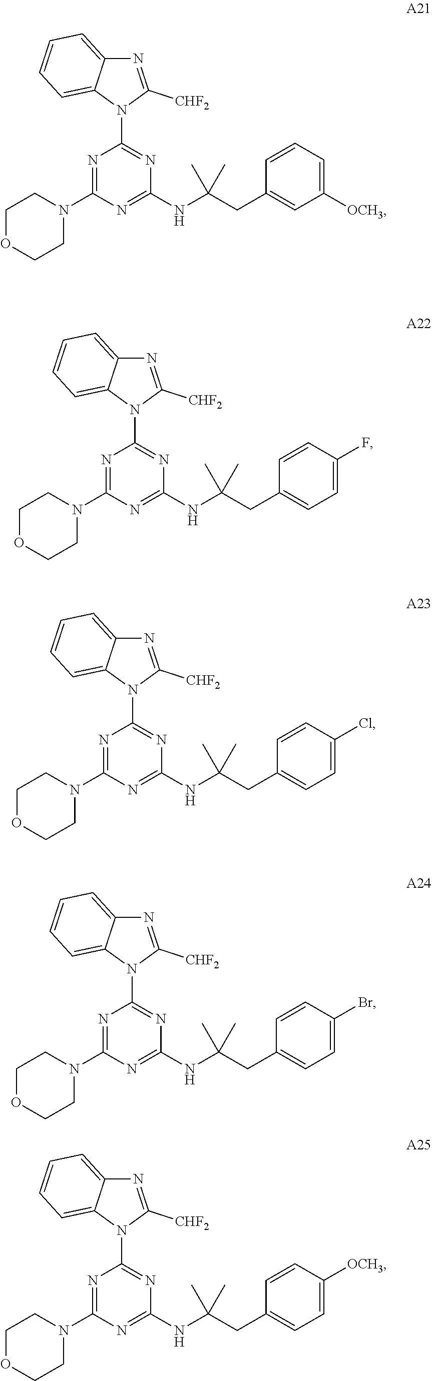

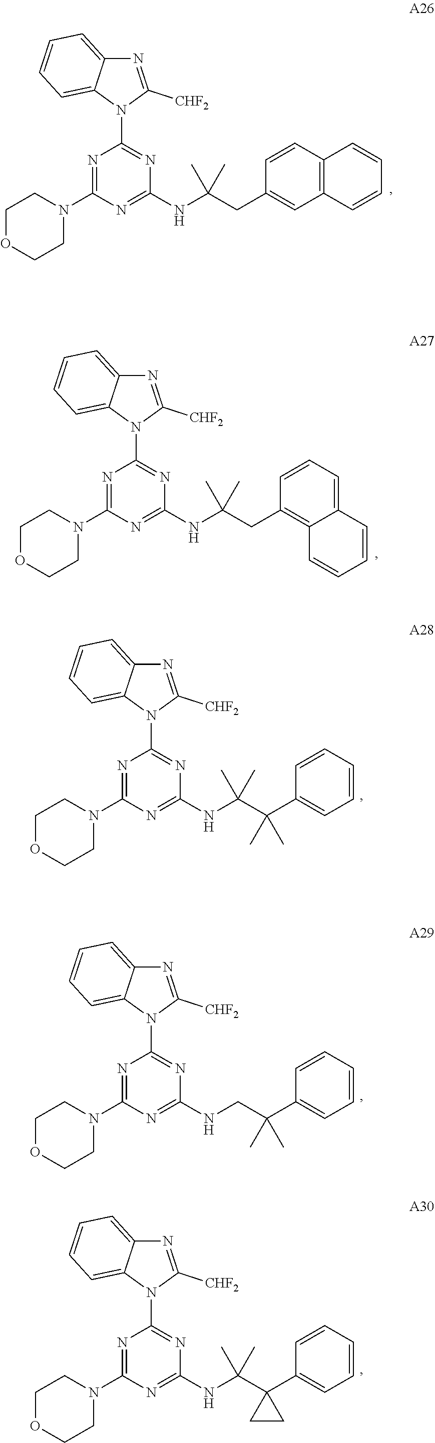 [SMILES: CC(C)(Cc1ccc2ccccc2c1)Nc1nc(N2CCOCC2)nc(-n2c(C(F)F)nc3ccccc32)n1 ; CC(C)(Cc1cccc2ccccc12)Nc1nc(N2CCOCC2)nc(-n2c(C(F)F)nc3ccccc32)n1 ; CC(C)(Nc1nc(N2CCOCC2)nc(-n2c(C(F)F)nc3ccccc32)n1)C(C)(C)c1ccccc1 ; CC(C)(CNc1nc(N2CCOCC2)nc(-n2c(C(F)F)nc3ccccc32)n1)c1ccccc1 ; CC(C)(Nc1nc(N2CCOCC2)nc(-n2c(C(F)F)nc3ccccc32)n1)C1(c2ccccc2)CC1]

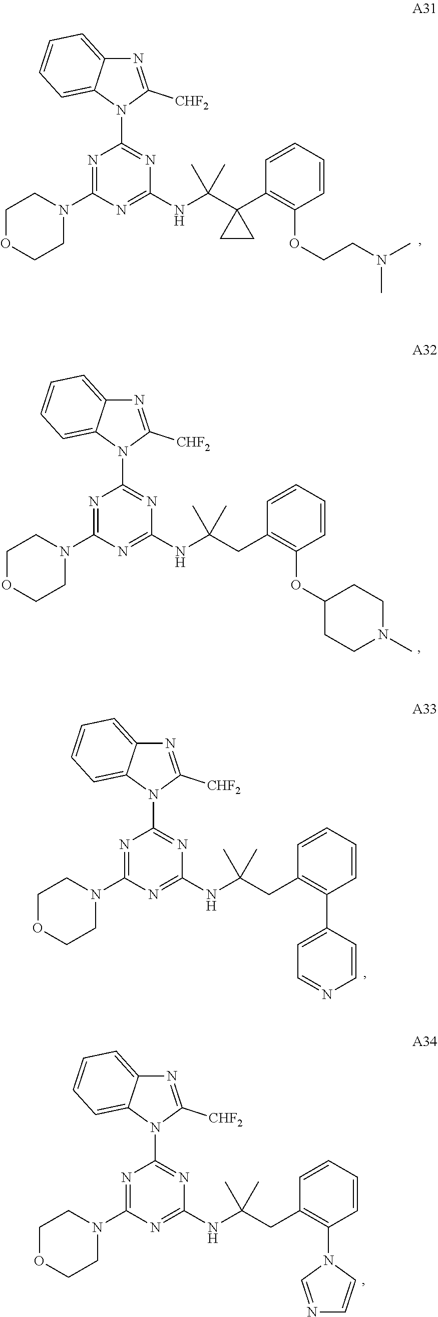 [SMILES: CN(C)CCOc1ccccc1C1(C(C)(C)Nc2nc(N3CCOCC3)nc(-n3c(C(F)F)nc4ccccc43)n2)CC1 ; CN1CCC(Oc2ccccc2CC(C)(C)Nc2nc(N3CCOCC3)nc(-n3c(C(F)F)nc4ccccc43)n2)CC1 ; CC(C)(Cc1ccccc1-c1ccncc1)Nc1nc(N2CCOCC2)nc(-n2c(C(F)F)nc3ccccc32)n1 ; CC(C)(Cc1ccccc1-n1ccnc1)Nc1nc(N2CCOCC2)nc(-n2c(C(F)F)nc3ccccc32)n1]

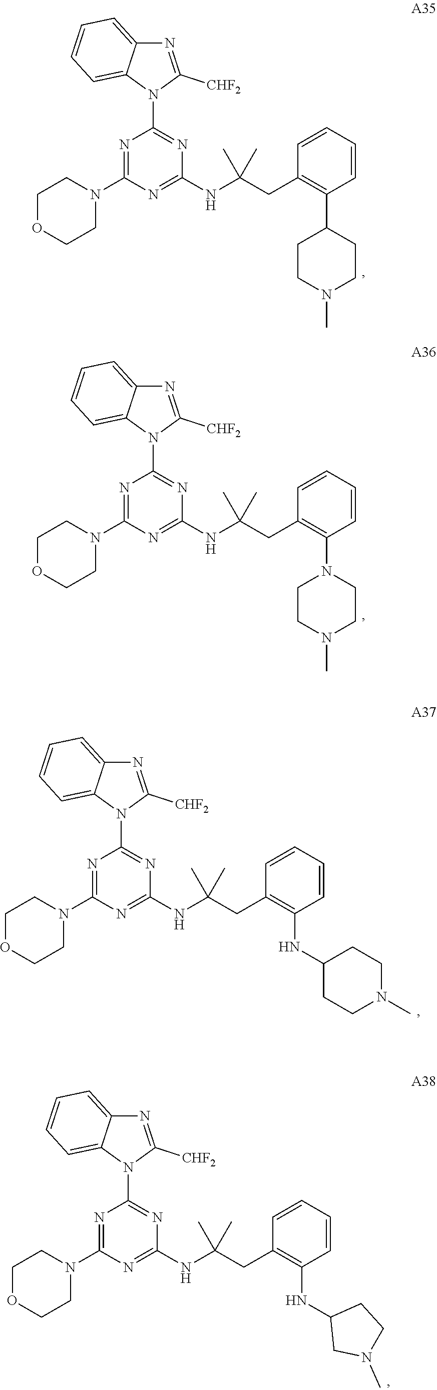 [SMILES: CN1CCC(c2ccccc2CC(C)(C)Nc2nc(N3CCOCC3)nc(-n3c(C(F)F)nc4ccccc43)n2)CC1 ; CN1CCN(c2ccccc2CC(C)(C)Nc2nc(N3CCOCC3)nc(-n3c(C(F)F)nc4ccccc43)n2)CC1 ; CN1CCC(Nc2ccccc2CC(C)(C)Nc2nc(N3CCOCC3)nc(-n3c(C(F)F)nc4ccccc43)n2)CC1 ; CN1CCC(Nc2ccccc2CC(C)(C)Nc2nc(N3CCOCC3)nc(-n3c(C(F)F)nc4ccccc43)n2)C1]

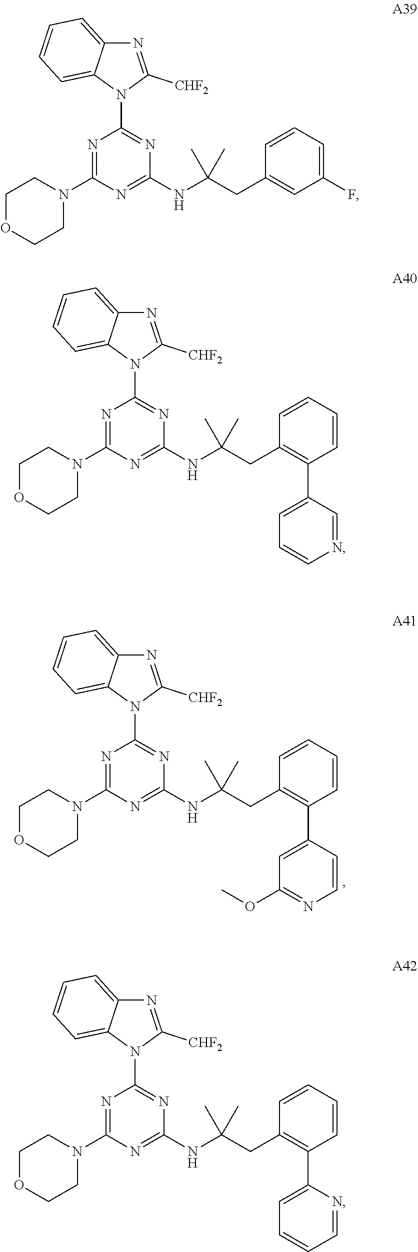 [SMILES: CC(C)(Cc1cccc(F)c1)Nc1nc(N2CCOCC2)nc(-n2c(C(F)F)nc3ccccc32)n1 ; CC(C)(Cc1ccccc1-c1cccnc1)Nc1nc(N2CCOCC2)nc(-n2c(C(F)F)nc3ccccc32)n1 ; COc1cc(-c2ccccc2CC(C)(C)Nc2nc(N3CCOCC3)nc(-n3c(C(F)F)nc4ccccc43)n2)ccn1 ; CC(C)(Cc1ccccc1-c1ccccn1)Nc1nc(N2CCOCC2)nc(-n2c(C(F)F)nc3ccccc32)n1]

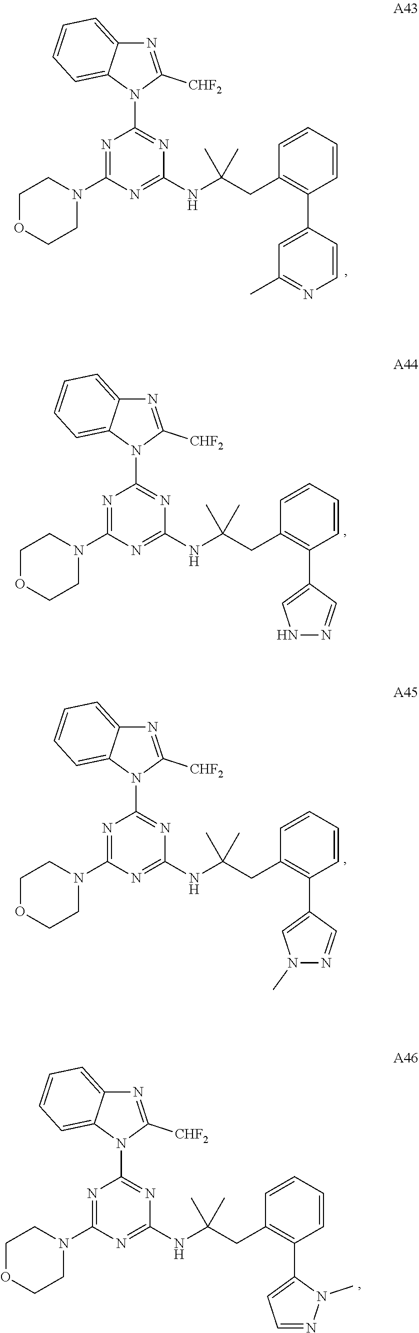 [SMILES: Cc1cc(-c2ccccc2CC(C)(C)Nc2nc(N3CCOCC3)nc(-n3c(C(F)F)nc4ccccc43)n2)ccn1 ; CC(C)(Cc1ccccc1-c1cn[nH]c1)Nc1nc(N2CCOCC2)nc(-n2c(C(F)F)nc3ccccc32)n1 ; Cn1cc(-c2ccccc2CC(C)(C)Nc2nc(N3CCOCC3)nc(-n3c(C(F)F)nc4ccccc43)n2)cn1 ; Cn1nccc1-c1ccccc1CC(C)(C)Nc1nc(N2CCOCC2)nc(-n2c(C(F)F)nc3ccccc32)n1]

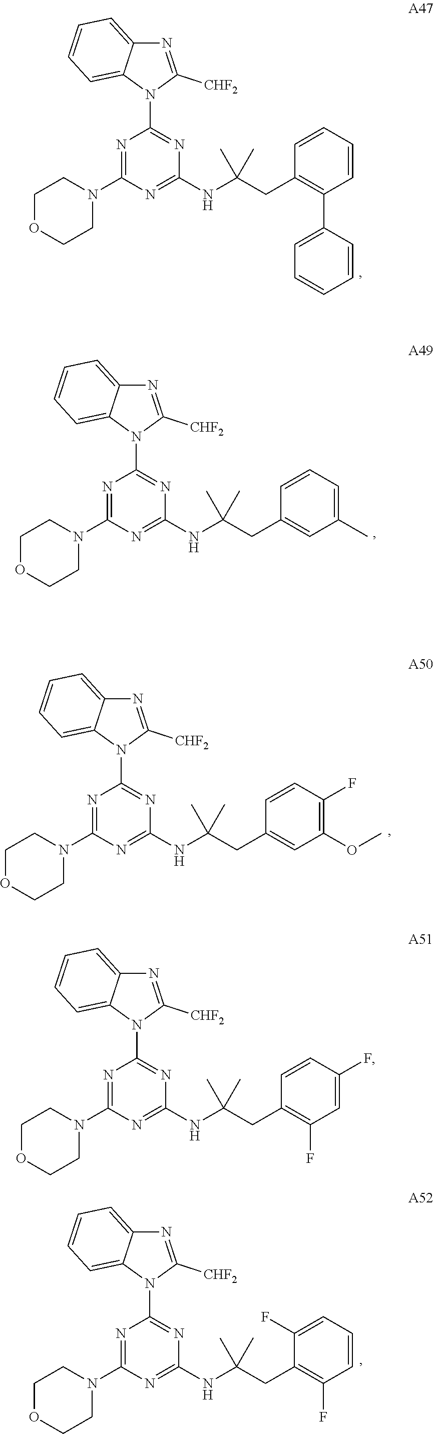 [SMILES: CC(C)(Cc1ccccc1-c1ccccc1)Nc1nc(N2CCOCC2)nc(-n2c(C(F)F)nc3ccccc32)n1 ; Cc1cccc(CC(C)(C)Nc2nc(N3CCOCC3)nc(-n3c(C(F)F)nc4ccccc43)n2)c1 ; COc1cc(CC(C)(C)Nc2nc(N3CCOCC3)nc(-n3c(C(F)F)nc4ccccc43)n2)ccc1F ; CC(C)(Cc1ccc(F)cc1F)Nc1nc(N2CCOCC2)nc(-n2c(C(F)F)nc3ccccc32)n1 ; CC(C)(Cc1c(F)cccc1F)Nc1nc(N2CCOCC2)nc(-n2c(C(F)F)nc3ccccc32)n1]

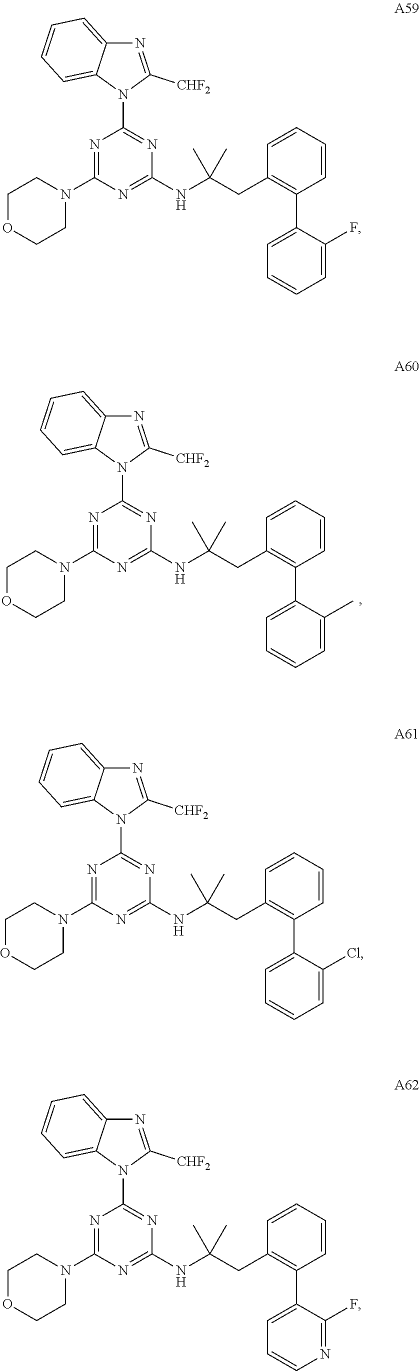 [SMILES: CC(C)(Cc1ccccc1-c1ccccc1F)Nc1nc(N2CCOCC2)nc(-n2c(C(F)F)nc3ccccc32)n1 ; Cc1ccccc1-c1ccccc1CC(C)(C)Nc1nc(N2CCOCC2)nc(-n2c(C(F)F)nc3ccccc32)n1 ; CC(C)(Cc1ccccc1-c1ccccc1Cl)Nc1nc(N2CCOCC2)nc(-n2c(C(F)F)nc3ccccc32)n1 ; CC(C)(Cc1ccccc1-c1cccnc1F)Nc1nc(N2CCOCC2)nc(-n2c(C(F)F)nc3ccccc32)n1]

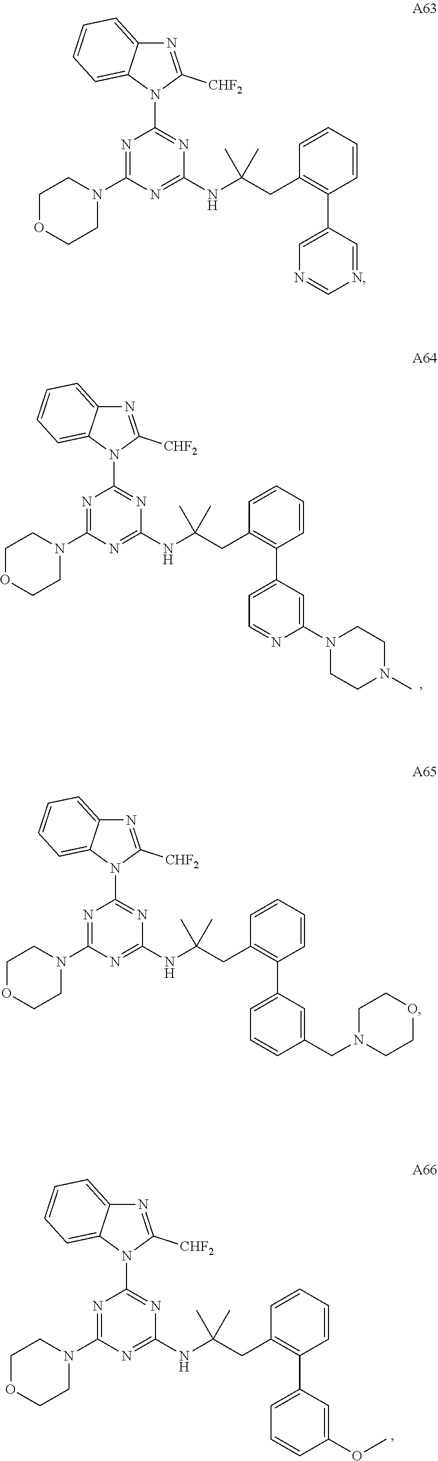 [SMILES: CC(C)(Cc1ccccc1-c1cncnc1)Nc1nc(N2CCOCC2)nc(-n2c(C(F)F)nc3ccccc32)n1 ; CN1CCN(c2cc(-c3ccccc3CC(C)(C)Nc3nc(N4CCOCC4)nc(-n4c(C(F)F)nc5ccccc54)n3)ccn2)CC1 ; CC(C)(Cc1ccccc1-c1cccc(CN2CCOCC2)c1)Nc1nc(N2CCOCC2)nc(-n2c(C(F)F)nc3ccccc32)n1 ; COc1cccc(-c2ccccc2CC(C)(C)Nc2nc(N3CCOCC3)nc(-n3c(C(F)F)nc4ccccc43)n2)c1]

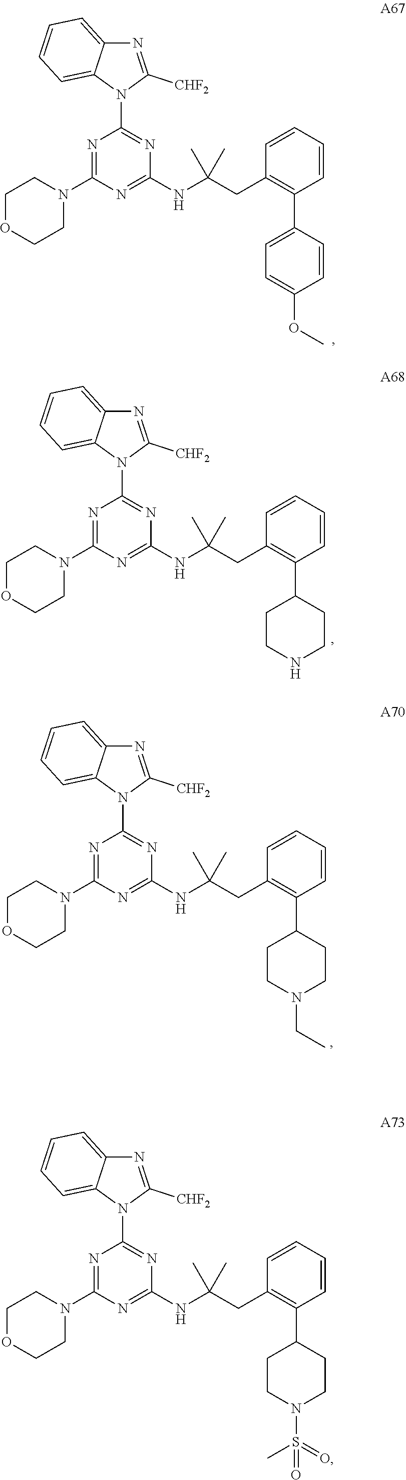 [SMILES: COc1ccc(-c2ccccc2CC(C)(C)Nc2nc(N3CCOCC3)nc(-n3c(C(F)F)nc4ccccc43)n2)cc1 ; CC(C)(Cc1ccccc1C1CCNCC1)Nc1nc(N2CCOCC2)nc(-n2c(C(F)F)nc3ccccc32)n1 ; CCN1CCC(c2ccccc2CC(C)(C)Nc2nc(N3CCOCC3)nc(-n3c(C(F)F)nc4ccccc43)n2)CC1 ; CC(C)(Cc1ccccc1C1CCN(S(C)(=O)=O)CC1)Nc1nc(N2CCOCC2)nc(-n2c(C(F)F)nc3ccccc32)n1]

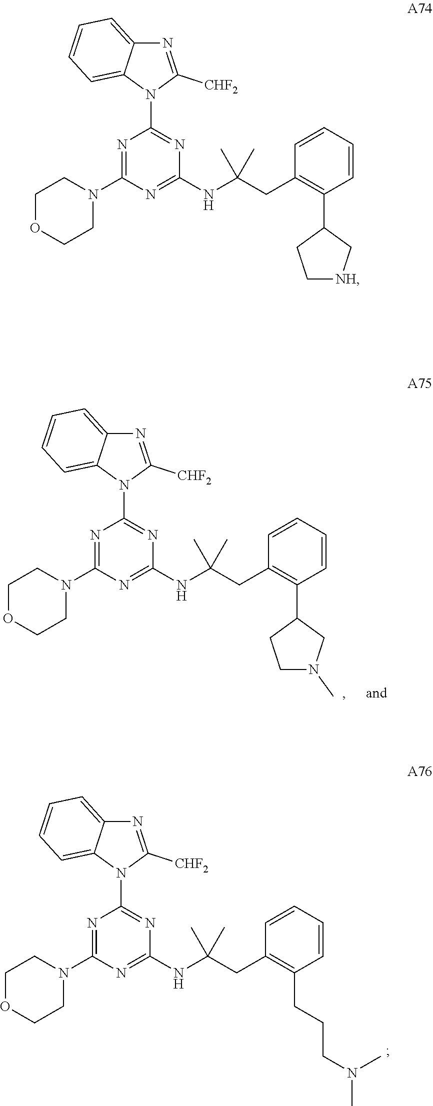 [SMILES: CC(C)(Cc1ccccc1C1CCNC1)Nc1nc(N2CCOCC2)nc(-n2c(C(F)F)nc3ccccc32)n1 ; CN1CCC(c2ccccc2CC(C)(C)Nc2nc(N3CCOCC3)nc(-n3c(C(F)F)nc4ccccc43)n2)C1 ; CN(C)CCCc1ccccc1CC(C)(C)Nc1nc(N2CCOCC2)nc(-n2c(C(F)F)nc3ccccc32)n1]

or an isotopic variant, pharmaceutically acceptable salt, solvate, hydrate, or prodrug thereof.

In one embodiment, the PI3K inhibitor is Compound A35, or an isotopic variant, pharmaceutically acceptable salt, solvate, hydrate, or prodrug thereof. In one embodiment, the PI3K inhibitor is Compound A36, or an isotopic variant, pharmaceutically acceptable salt, solvate, hydrate, or prodrug thereof. In one embodiment, the PI3K inhibitor is Compound A68, or an isotopic variant, pharmaceutically acceptable salt, solvate, hydrate, or prodrug thereof. In one embodiment, the PI3K inhibitor is Compound A70, or an isotopic variant, pharmaceutically acceptable salt, solvate, hydrate, or prodrug thereof. In one embodiment, the PI3K inhibitor is Compound A37, or an isotopic variant, pharmaceutically acceptable salt, solvate, hydrate, or prodrug thereof. In one embodiment, the PI3K inhibitor is Compound A38, or an isotopic variant, pharmaceutically acceptable salt, solvate, hydrate, or prodrug thereof. In one embodiment, the PI3K inhibitor is Compound A41, or an isotopic variant, pharmaceutically acceptable salt, solvate, hydrate, or prodrug thereof. In one embodiment, the PI3K inhibitor is Compound A42, or an isotopic variant, pharmaceutically acceptable salt, solvate, hydrate, or prodrug thereof. In one embodiment, the PI3K inhibitor is Compound A43, or an isotopic variant, pharmaceutically acceptable salt, solvate, hydrate, or prodrug thereof. In one embodiment, the PI3K inhibitor is Compound A44, or an isotopic variant, pharmaceutically acceptable salt, solvate, hydrate, or prodrug thereof. In one embodiment, the PI3K inhibitor is Compound A62, or an isotopic variant, pharmaceutically acceptable salt, solvate, hydrate, or prodrug thereof. In one embodiment, the PI3K inhibitor is Compound A63, or an isotopic variant, pharmaceutically acceptable salt, solvate, hydrate, or prodrug thereof. In one embodiment, the PI3K inhibitor is Compound A64, or an isotopic variant, pharmaceutically acceptable salt, solvate, hydrate, or prodrug thereof. In one embodiment, the PI3K inhibitor is Compound A65, or an isotopic variant, pharmaceutically acceptable salt, solvate, hydrate, or prodrug thereof. In one embodiment, the PI3K inhibitor is Compound A66, or an isotopic variant, pharmaceutically acceptable salt, solvate, hydrate, or prodrug thereof. In one embodiment, the PI3K inhibitor is Compound A67, or an isotopic variant, pharmaceutically acceptable salt, solvate, hydrate, or prodrug thereof.

Synthesis of compounds of any of the Formulae provided herein e.g., Formulae (I), (II), (VII), (IX), (X), (XI), (XVI), is described in U.S. Pat. No. 9,056,852 B2, which is incorporated by reference for such disclosure.

### CD20 Inhibitors

Described herein in some embodiments are PI3K inhibitors in combination with CD20 inhibitors useful for treating relapsed cancer patients identified as early progressors.

B lymphocytes are the origin of humoral immunity, represent a substantial portion of hematopoietic malignancies, and contribute to autoimmunity. Consequently, cell surface molecules expressed by B cells and their malignant counterparts are important targets for immunotherapy. CD20, a B cell-specific member of the MS4A gene family, is expressed on the surface of immature and mature B cells and their malignant counterparts.

A limited analysis of CD20 transcripts in mouse cell lines and tissues suggests that mouse CD20 is also B cell-specific. Both human and mouse CD20 cDNAs encode a membrane-embedded protein with hydrophobic regions of sufficient length to pass through the membrane four times. Mouse and human CD20 are well conserved (73%) in amino acid sequence, particularly the transmembrane and long amino- and carboxyl-terminal cytoplasmic domains. The cytoplasmic domains are serine- and threonine-rich with multiple consensus sequences for phosphorylation. Human CD20 is not glycosylated, but three isoforms (33-, 35- and 37,000 Mr) result from the differential phosphorylation of a single protein on different serine and threonine residues.

CD20 plays a role in the regulation of human B cell activation, proliferation, and Ca²⁺ transport. Antibody ligation of CD20 can generate transmembrane signals that result in enhanced CD20 phosphorylation, induction of c-myc and B-myb oncogene expression, induced serine/threonine and tyrosine phosphorylation of cellular proteins, increased CD18, CD58 and MHC class II molecule expression, and protein tyrosine kinase activation that induces B cell adhesion. CD20 ligation promotes transmembrane Ca²⁺ transport, but does not usually lead to increased intracellular calcium ([Ca²⁺]ᵢ)₃ levels, except after extensive crosslinking. Antibody binding to CD20 inhibits B cell progression from the G1 phase into the S/G2+M stages of cell cycle following mitogen stimulation, and inhibits mitogen-induced B cell differentiation and antibody secretion. Extensive CD20 cross-linking can also influence apoptosis. These divergent observations may be explained in part by the finding that CD20 is a component of an oligomeric complex that forms a membrane transporter or Ca²⁺ ion channel that is activated during cell cycle progression. Despite this, B cell development and function in a line of CD20-deficient (CD20-/−) mice is reported to be normal.

The majority of human B cell-lineage malignancies express CD20. Chimeric or radiolabeled monoclonal antibody-based therapies directed against CD20 have been used for B cell malignancies such as non-Hodgkin's lymphoma.

Any suitable CD20 inhibitor may be used in combination with a PI3K inhibitor described herein. In some embodiments, the CD20 inhibitor is an antagonist of CD20. In some embodiments, the CD20 inhibitor is an antibody, variant, or biosimilar thereof. In some embodiments, the CD20 inhibitor is a monoclonal antibody.

Some embodiments provided herein describe a pharmaceutical compositions or methods for using the pharmaceutical compositions comprising a PI3K inhibitor described herein in combination with a CD20 inhibitor. CD20 inhibitors for use in pharmaceutical compositions and methods provided herein include but are not limited to ofatumumab, obinutuzumab, rituximab, ocaratuzumab, ocrelizumab, tositumomab, ibritumomab tiuxetan, tisotumab vedotin, ublituximab, TRU-015, veltuzumab, BTCT4465A (RG7828), EDC9, MT-3724, BLX-301, 1 F5, ATCC deposit HB-96450, BM-ca, C2H7, PR0131921, BVX-20, MEDI-522, or a variant or biosimilar thereof, or combinations thereof. In some embodiments, the CD20 inhibitor for use in pharmaceutical compositions and methods provided herein is ofatumumab, obinutuzumab, rituximab, ocaratuzumab, ocrelizumab, tositumomab, ibritumomab tiuxetan, tisotumab vedotin, ublituximab, TRU-015, veltuzumab, BTCT4465A (RG7828), EDC9, MT-3724, or a variant or biosimilar thereof, or combinations thereof. In some embodiments, the CD20 inhibitor for use in pharmaceutical compositions and methods provided herein is ofatumumab, obinutuzumab, rituximab, ocaratuzumab, ocrelizumab, tositumomab, ibritumomab tiuxetan, tisotumab vedotin, ublituximab, veltuzumab, or a variant or biosimilar thereof, or combinations thereof. In some embodiments, the CD20 inhibitor for use in pharmaceutical compositions and methods provided herein is obinutuzumab or rituximab, or a variant or biosimilar thereof, or combinations thereof.

In some embodiments, the CD20 inhibitor is ofatumumab, an ofatumumab variant, or an ofatumumab biosimilar. In some embodiments, the CD20 inhibitor is obinutuzumab, an obinutuzumab variant, or an obinutuzumab biosimilar. In some embodiments, the CD20 inhibitor is rituximab, a rituximab variant, or a rituximab biosimilar. In some embodiments, the rituximab biosimilar is CT-P10, Reditux®, ABP 798, AcellBia, BI 695500, Maball, JHL1101, Novex, MabionCD20, PF-05280586, Kikuzubam, SAIT101, GP 2013, HLX01, CMAB304, BT-D004, AP-052 or TL-011. In some embodiments, the CD20 inhibitor is ocaratuzumab, an ocaratuzumab variant, or an ocaratuzumab biosimilar. In some embodiments, the CD20 inhibitor is ocrelizumab, an ocrelizumab variant, or an ocrelizumab biosimilar. In some embodiments, the CD20 inhibitor is tositumomab, a tositumomab variant, or a tositumomab biosimilar. In some embodiments, the CD20 inhibitor is ibritumomab tiuxetan, an ibritumomab tiuxetan variant, or an ibritumomab tiuxetan biosimilar. In some embodiments, the CD20 inhibitor is tisotumab vedotin, a tisotumab vedotin variant, or a tisotumab vedotin biosimilar. In some embodiments, the CD20 inhibitor is ublituximab, an ublituximab variant, or an ublituximab biosimilar. In some embodiments, the CD20 inhibitor is TRU-015, a TRU-015 variant, or a TRU-015 biosimilar. In some embodiments, the CD20 inhibitor is veltuzumab, a veltuzumab variant, or a veltuzumab biosimilar. In some embodiments, the CD20 inhibitor is BTCT4465A (RG7828), a BTCT4465A (RG7828) variant, or a BTCT4465A (RG7828) biosimilar. In some embodiments, the CD20 inhibitor is EDC9, an EDC9 variant, or an EDC9 biosimilar. In some embodiments, the CD20 inhibitor is MT-3724, a MT-3724 variant, or a MT-3724 biosimilar.

Uses for Treating FL Patients with Early Progression of Disease

In an aspect provided herein is a method for treating a patient with follicular lymphoma (FL) having early disease progression after immuochemotherapy treatment, comprising administering to the patient an effective amount of a PI3K inhibitor of Formula (I). Also provided herein, is a method of preventing relapse in a patient with follicular lymphoma having early disease progression after immunochemotherapy treatment, comprising administering to the patient an effective amount of a PI3K inhibitor of Formula (I). In some embodiments provided herein is a method for achieving and retaining partial cancer remission a patient with follicular lymphoma having early disease progression after immuochemotherapy treatment, comprising administering to the patient an effective amount of a PI3K inhibitor of Formula (I). In some embodiments provided herein is a method for achieving and retaining complete cancer remission in a patient with follicular lymphoma having early disease progression after immuochemotherapy treatment, comprising administering to the patient an effective amount of a PI3K inhibitor of Formula (I). In some embodiments, the methods described above further comprise administering to the patient an effective amount of a CD20 inhibitor. In some embodiments of the methods described above, the patient has progression of disease within 24 months of treatment of FL. In some embodiments of the methods described above, the patient has progression of disease after initiation of treatment of FL with immunochemotherapy. In some embodiments of the methods described above, the patient has progression of disease within 24 months after initiation of treatment of FL with immunochemotherapy. In some embodiments of the methods described above, the immunochemotherapy treatment is first-line. In some embodiments of the methods described above, the immunochemotherapy treatment is subsequent to first-line treatment.

In some embodiments, the methods described herein avoid and/or reduce adverse or unwanted side effects associated with the use of the PI3K inhibitor or immunochemotherapy. In some embodiments, the methods described herein avoid, reduce, or minimize the risk of death due to infections associated with PI3K inhibitor treatment or immunochemotherapy. In some embodiments, the methods described herein avoid, reduce, or minimize infections, neutropenia, diarrhea/colitis, elevated liver transaminases (alanine aminotransferase/aspartate aminotransferase >5× upper limit of normal), pneumonitis, rash, hepatic impairment, renal impairment, pyrexia, or increased triglycerides, or a combination thereof in patients receiving the treatment described herein. In certain embodiments, the methods described herein avoid, reduce, or minimize the incidence of infection. In certain embodiments, the methods described herein avoid, reduce, or minimize the incidence of neutropenia. In certain embodiments, the methods described herein avoid, reduce, or minimize the incidence of diarrhea/colitis. In certain embodiments, the methods described herein avoid, reduce, or minimize the incidence of elevated liver transaminases. In certain embodiments, the methods described herein avoid, reduce, or minimize the incidence of pneumonitis. In certain embodiments, the methods described herein avoid, reduce, or minimize the incidence of a rash. In certain embodiments, the methods described herein avoid, reduce, or minimize the incidence of hepatic impairment or renal impairment. In certain embodiments, the methods described herein avoid, reduce, or minimize the incidence of pyrexia. In certain embodiments, the methods described herein avoid, reduce, or minimize the incidence of increased triglycerides. In certain embodiments, the methods described herein avoid, reduce, or minimize enterocolitis (manifested as diarrhea), cutaneous toxicities, liver toxicity (manifested as elevation of transaminases), pulmonary toxicity (manifested as non-infectious pneumonitis), infections, or combinations thereof.

In some embodiments, the methods described herein provides a high objective response rate (ORR) as determined by tumor assessment from radiological tests and/or physical examination. In some embodiments, the methods described herein provide a durable response (DR) and/or increased durable response rate (DRR; a continuous response [complete or partial objective response] beginning within 12 months of treatment and lasting ≥6 months) in the subject or patient. In some embodiments, the methods described herein provide complete remission. In some embodiments, the methods described herein provide a better response compared to the monotherapy treatment of a compound of Formula (I) and/or a CD20 inhibitor. In some embodiments, the methods described herein provide complete remission beginning within 12 months of treatment and lasting ≥6 months. In some embodiments, the methods described herein provide a complete response (CR) and/or no evidence of disease (NED) beginning within 12 months of treatment and lasting ≥6 months.

In some embodiments of a method of treating follicular lymphoma (FL) including relapsed or refractory FL, the discontinuation rate due to adverse events is less than 25%, less than 20%, less than 15%, less than 10%, less than 8%, less than 5%.

The “discontinuation rate” is defined as the number of subjects who discontinue the study drugs prior to the study completion divided by the number of subjects treated.

In some embodiments, the discontinuation rate due to adverse events is less than 25%, less than 20%, less than 15%, less than 10%, less than 8%, less than 5%. In some embodiments, the discontinuation rate due to adverse events is less than 25%. In some embodiments, the discontinuation rate due to adverse events is less than 20%. In some embodiments, the discontinuation rate due to adverse events is less than 15%. In some embodiments, the discontinuation rate due to adverse events is less than 10%. In some embodiments, the discontinuation rate due to adverse events is less than 8%. In some embodiments, the discontinuation rate due to adverse events is about 4%.

In some embodiments, the discontinuation rate due to adverse events when the subjects are administered a compound of Formula (I), or an isotopic variant thereof or a pharmaceutically acceptable salt, solvate, hydrate, or prodrug is less for subjects on an intermittent dosing schedule (IS) than the discontinuation rate observed for subjects on a continuous dosing schedule (CS).

In certain embodiments, provided herein are methods for treating or preventing a disease comprising administering an effective amount of a compound of Formula (I), or an isotopic variant thereof or a pharmaceutically acceptable salt, solvate, hydrate, or prodrug. In some embodiments, the compound of Formula (I) is Compound A35 or an isotopic variant, pharmaceutically acceptable salt, solvate, hydrate, or prodrug thereof. In some embodiments, the compound of Formula (I) is Compound A36 or an isotopic variant, pharmaceutically acceptable salt, solvate, hydrate, or prodrug thereof. In some embodiments, the compound of Formula (I) is Compound A68 or an isotopic variant, pharmaceutically acceptable salt, solvate, hydrate, or prodrug thereof. In some embodiments, the compound of Formula (I) is Compound A70 or an isotopic variant, pharmaceutically acceptable salt, solvate, hydrate, or prodrug thereof. In some embodiments, the compound of Formula (I) is Compound A37 or an isotopic variant, pharmaceutically acceptable salt, solvate, hydrate, or prodrug thereof. In some embodiments, the compound of Formula (I) is Compound A38 or an isotopic variant, pharmaceutically acceptable salt, solvate, hydrate, or prodrug thereof. In some embodiments, the compound of Formula (I) is Compound A41 or an isotopic variant, pharmaceutically acceptable salt, solvate, hydrate, or prodrug thereof. In some embodiments, the compound of Formula (I) is Compound A42 or an isotopic variant, pharmaceutically acceptable salt, solvate, hydrate, or prodrug thereof. In some embodiments, the compound of Formula (I) is Compound A43 or an isotopic variant, pharmaceutically acceptable salt, solvate, hydrate, or prodrug thereof. In some embodiments, the compound of Formula (I) is Compound A44 or an isotopic variant, pharmaceutically acceptable salt, solvate, hydrate, or prodrug thereof. In some embodiments, the compound of Formula (I) is Compound A62 or an isotopic variant, pharmaceutically acceptable salt, solvate, hydrate, or prodrug thereof. In some embodiments, the compound of Formula (I) is Compound A63 or an isotopic variant, pharmaceutically acceptable salt, solvate, hydrate, or prodrug thereof. In some embodiments, the compound of Formula (I) is Compound A64 or an isotopic variant, pharmaceutically acceptable salt, solvate, hydrate, or prodrug thereof. In some embodiments, the compound of Formula (I) is Compound A65 or an isotopic variant, pharmaceutically acceptable salt, solvate, hydrate, or prodrug thereof. In some embodiments, the compound of Formula (I) is Compound A66 or an isotopic variant, pharmaceutically acceptable salt, solvate, hydrate, or prodrug thereof. In some embodiments, the compound of Formula (I) is Compound A67 or an isotopic variant, pharmaceutically acceptable salt, solvate, hydrate, or prodrug thereof.

In some embodiments, the methods further comprise administering the patient or subject with a CD20 inhibitor, wherein the CD20 inhibitor is ofatumumab, obinutuzumab, rituximab, ocaratuzumab, ocrelizumab, tositumomab, ibritumomab tiuxetan, tisotumab vedotin, ublituximab, TRU-015, veltuzumab, BTCT4465A (RG7828), EDC9, MT-3724, or a variant or biosimilar thereof. In some embodiments, the CD20 inhibitor is rituximab, obinutuzumab, ofatumumab, ocaratuzumab, tositumomab, ibritumomab tiuxetan, ublituximab, EDC9, MT-3724, or a variant, or biosimilar thereof. In some embodiments, the CD20 inhibitor is rituximab, or a variant or biosimilar thereof.

Resistant, relapsed or refractory refers to when a cancer that has a reduced responsiveness to a treatment, e.g., up to the point where the cancer does not respond to treatment. The cancer can be resistant at the beginning of treatment, or it may become resistant during treatment. The term “refractory” can refer to a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

### Dosages and Dosing Regimens

In some embodiments, the methods provided herein comprise administering a compound of Formula (I), or an isotopic variant thereof; or a pharmaceutically acceptable salt, solvate, hydrate, or prodrug thereof.

In certain embodiments, the method further comprises administering a CD20 inhibitor to the patient simultaneously or sequentially by the same or different routes of administration. In certain embodiments, the compound of Formula (I), or an isotopic variant thereof; or a pharmaceutically acceptable salt, solvate, hydrate, or prodrug thereof and a CD20 inhibitor is administered simultaneously, at essentially the same time, or sequentially. If administration takes place sequentially, the CD20 inhibitor may be administered before or after administration of a compound of Formula (I), or an isotopic variant thereof; or a pharmaceutically acceptable salt, solvate, hydrate, or prodrug thereof. In some embodiments, the CD20 inhibitor is administered before administration of a compound of Formula (I), or an isotopic variant thereof; or a pharmaceutically acceptable salt, solvate, hydrate, or prodrug thereof. In some embodiments, the CD20 inhibitor is administered simultaneously with administration of a compound of Formula (I), an isotopic variant thereof; or a pharmaceutically acceptable salt, solvate, hydrate, or prodrug thereof. In some embodiments, the CD20 inhibitor is administered after the administration of a compound of Formula (I), an isotopic variant thereof; or a pharmaceutically acceptable salt, solvate, hydrate, or prodrug thereof.

In some instances, the methods described herein further comprise administering the PI3K inhibitor in combination with CD20 inhibitor to the subject or patient in need thereof in multiple cycles repeated on a regular schedule with periods of rest in between each cycle. For example, in some instances, treatment is given for one week followed by three weeks of rest is one treatment cycle.

In some instances, a cycle comprises administration of the PI3K inhibitor at the same time as administration of the CD20 inhibitor. In some instances, the PI3K inhibitor and the CD20 inhibitor are administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days.

In some instances, a cycle comprises administration of the PI3K inhibitor first followed by administration of the CD20 inhibitor second. In some instances, the PI3K inhibitor is administered for about 1 day, about 2 days, about 3 days, about 4 days, about 5 days, about 6 days, about 7 days, about 8 days, about 9 days, about 10 days, about 11 days, about 12 days, about 13 days, or about 14 days followed by administration of the CD20 inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days.

In some instances, a cycle comprises administration of the PI3K inhibitor first followed by concurrent administration of the CD20 inhibitor. In some instances, the PI3K inhibitor is first administered for about 1 day, about 2 days, about 3 days, about 4 days, about 5 days, about 6 days, about 7 days, about 8 days, about 9 days, about 10 days, about 11 days, about 12 days, about 13 days, or about 14 days followed by the concurrent administration of the CD20 inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days. In some instances, the PI3K inhibitor is first administered for about 1 day, about 2 days, about 3 days, about 4 days, about 5 days, about 6 days, or about 7 days followed by the concurrent administration of the CD20 inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days. In some instances, the PI3K inhibitor is first administered for about 7 days followed by the concurrent administration of the CD20 inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days. In some instances, the PI3K inhibitor is first administered for about 7 days followed by the concurrent administration of the CD20 inhibitor for about 10 days, about 11 days, about 12 days, about 13 days, or about 14 days.

In some instances, a cycle comprises administration of the PI3K inhibitor only. In some instances, the PI3K inhibitor is administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days.

In some instances, the method for multiple cycle chemotherapy comprises the administration of a second cycle within about 60 days or about 3 months. In some instances, the method for multiple cycle chemotherapy comprises the administration of a second cycle within 50 days. In another instance, the second cycle is administered within 45, 40, 35, 30, 25, 21, 20, 15, 14, 10, 9, 8, 7, 6, 5, 4, 3, 2 or 1 day(s) of the first cycle. In some embodiments, the administration of any additional cycles is within 50 days of the previous cycle. In some embodiments, the administration of any additional cycles is within 10 days of the previous cycle. In some embodiments, the administration of any additional cycles is within 9 days of the previous cycle. In some embodiments, the administration of any additional cycles is within 8 days of the previous cycle. In some embodiments, the administration of any additional cycles is within 7 days of the previous cycle. In some embodiments, the administration of any additional cycles is within 6 days of the previous cycle. In some embodiments, the administration of any additional cycles is within 5 days of the previous cycle. In some embodiments, the administration of any additional cycles is within 4 days of the previous cycle. In some embodiments, the administration of any additional cycles is within 3 days of the previous cycle. In some embodiments, the administration of any additional cycles is within 2 days of the previous cycle. In some embodiments, the administration of any additional cycles is within 1 day of the previous cycle. In another embodiment, the additional cycle is administered within 45, 40, 35, 30, 25, 21, 20, 15, 14, 10, 9, 8, 7, 6, 5, 4, 3, 2, or 1 days of the previous cycle.

The length of a treatment cycle depends on the treatment being given. In some embodiments, the length of a treatment cycle ranges from two to six weeks. In some embodiments, the length of a treatment cycle ranges from four to six weeks. In some embodiments, the length of a treatment cycle is 28 days. In some embodiments, the length of a treatment cycle is 56 days. In some embodiments, a treatment cycle lasts one, two, three, or four weeks. In some embodiments, a treatment cycle lasts four weeks. The number of treatment doses scheduled within each cycle also varies depending on the drugs being given.

In certain instances, the compound of Formula (I), or an isotopic variant thereof; or a pharmaceutically acceptable salt, solvate, hydrate, or prodrug thereof, is administered to the subject on a 28-day cycle. In some embodiments, the compound of Formula (I), or an isotopic variant thereof; or a pharmaceutically acceptable salt, solvate, hydrate, or prodrug thereof, is administered to the subject for at least one 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two 28-day cycles.

In certain embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for a period of up to about 7 days. In some embodiments, the days over which the compound of Formula (I), or an enantiomer, a mixture of enantiomers, a mixture of two or more diastereomers, or an isotopic variant thereof; or a pharmaceutically acceptable salt, solvate, hydrate, or prodrug thereof are intermittent. In some embodiments, administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof for about 7 consecutive days in a 28-day cycle.

In some embodiments, the method comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle. In some embodiments, the IS avoids or reduces adverse or unwanted side effects associated with the use of the PI3K inhibitor, such as enterocolitis (manifested as diarrhea), cutaneous toxicities, liver toxicity (manifested as elevation of transaminases), pulmonary toxicity (manifested as non-infectious pneumonitis), and infections. In some embodiments, the IS avoids or reduces enterocolitis, rash, transaminitis, or combinations thereof.

In some embodiments, the method comprises administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof on a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for 28 consecutive days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on continuous dosing schedule (CS). In some embodiments, the continuous dosing schedule (CS), comprises once daily administration of compound of Formula (I), or an enantiomer, a mixture of enantiomers, a mixture of two or more diastereomers, or an isotopic variant thereof; or a pharmaceutically acceptable salt, solvate, hydrate, or prodrug thereof to the subject for 28 consecutive days in a 28-day cycle.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two 28-day cycles. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least three 28-day cycles. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered daily to the subject on a 28-day continuous schedule until progression of disease or intolerable toxicity.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 intermittent days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 consecutive days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 consecutive days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS). In some embodiments, the intermittent dosing schedule (IS), comprises once daily administration of compound of Formula (I), or an enantiomer, a mixture of enantiomers, a mixture of two or more diastereomers, or an isotopic variant thereof; or a pharmaceutically acceptable salt, solvate, hydrate, or prodrug thereof to the subject for 7 consecutive days followed by 21 days without treatment in a 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least three 28-day cycles, wherein: the first two 28-day cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for two 28-day cycles; and the third 28-day cycle comprises an intermittent dosing schedule (IS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for the first 7 consecutive days of the 28-day cycle. In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least three cycles, wherein: the first two cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for two cycles; and the subsequent cycle(s) comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for only the first 7 consecutive days in each subsequent cycle. In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for four or more 28-day cycles, wherein: the first two or three 28-day cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for three or more 28-day cycles; and the subsequent 28-day cycle(s) comprise(s) an intermittent dosing schedule (IS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for the first 7 consecutive days of the 28-day cycle.

In certain instances, CS refers to continuous daily dosing to a subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily on a 28-day schedule with no switch to IS. In certain instances, CS refers to continuous daily dosing to a subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily on a 28-day schedule for four or more cycles followed by a switch to IS (i.e., late switch to IS). In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof is administered to a subject on an intermittent dosing schedule (IS) until progression of disease. In some embodiments, upon progression of disease, the subject resumes continuous daily dosing (CS) of the compound of Formula (I), or an enantiomer, a mixture of enantiomers, a mixture of two or more diastereomers, or an isotopic variant thereof; or a pharmaceutically acceptable salt, solvate, hydrate, or prodrug thereof.

In certain instances, the method comprises a continuous daily dosing schedule (CS) for at least two CS 28-day cycles, followed by an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle after the at least two CS 28-day cycles. In some embodiments, the dosing schedule avoids or reduces adverse or unwanted side effects associated with the use of the PI3K inhibitor, such as enterocolitis (manifested as diarrhea), cutaneous toxicities, liver toxicity (manifested as elevation of transaminases), pulmonary toxicity (manifested as non-infectious pneumonitis), and infections. In some embodiments, the dosing schedule avoids or reduces enterocolitis, rash, transaminitis, or combinations thereof.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) until disease progression occurs.

In some or additional embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered daily to the subject on a continuous dosing schedule (CS) after disease progression occurs on an intermittent dosing schedule (IS).

In some embodiments, the methods of treatment and dosing regimens and schedules described herein improve the frequency, severity and time to onset of the adverse events (AEs) associated with PI3K delta inhibitors. In some embodiments, the methods of treatment and dosing regimens and schedules described herein, including IS dosing regimens, result in partial or complete remission.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) resulting in disease stabilization. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) resulting in disease regression. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) resulting in an objective response. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) until disease stabilization is no longer observed. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) until disease progression is observed.

In certain instances of the treatment regimen comprising administration of the compound of Formula (I), or an enantiomer, a mixture of enantiomers, a mixture of two or more diastereomers, or an isotopic variant thereof; or a pharmaceutically acceptable salt, solvate, hydrate, or prodrug thereof for two cycles of continuous daily administration (CS) followed by daily administration for only the first seven days of each subsequent (IS) cycle, the CS and IS cycles are 28-day cycles, wherein the IS cycle is repeated until disease regression is no longer observed. In some or additional embodiments, if disease progression is observed in the subject, the subject resumes the 28-day cycles of continuous daily administration (CS) until disease regression or stabilization are observed.

In certain instances of the treatment regimen comprising administration of the compound of Formula (I), or an enantiomer, a mixture of enantiomers, a mixture of two or more diastereomers, or an isotopic variant thereof; or a pharmaceutically acceptable salt, solvate, hydrate, or prodrug thereof for two 28-day cycles of continuous daily administration (CS) followed by daily administration for only the first seven days of each subsequent (IS) 28-day cycle; wherein disease regression or stabilization is no longer observed in the subject on the intermittent dosing schedule (IS) cycle, the subject resumes 28-day cycles of continuous daily administration (CS) until disease regression or stabilization are observed.

In some embodiments, the methods of treatment and dosing regimens and schedules described herein provide an efficaious and tolerable treatment of cancer. In some embodiments, the methods of treatment and dosing regimens and schedules described herein improve the frequency, severity and time to onset of the adverse events (AEs) associated with PI3K delta inhibitors. In some embodiments, the methods of treatment and dosing regimens and schedules described herein, including IS dosing regimens, result in partial or complete remission.

In some instances, the method for the administration of multiple compounds comprises administering compounds within 48 hours or less of each other. In some embodiments administration occurs within 24 hours, 12 hours, 6 hours, 3 hours, 1 hour, or 15 minutes. In some instances, the compounds are administered simultaneously. One example of simultaneous administration is the injection of one compound immediately before, after, or during the oral administration of the second compound, immediately referring to a time less than about 5 minutes.

In some instances, the method for the administration of multiple compounds occurs in a sequential order, wherein the PI3K inhibitor is administered before the CD20 inhibitor. In another instance, the CD20 inhibitor is administered before the PI3K inhibitor.

In some instances, the method for administering the PI3K inhibitor is oral and the method for administering the CD20 inhibitor is by injection. In some instances, the method for administering the PI3K inhibitor is by inhalation and the method for administering the CD20 inhibitor is by injection. In some instances, the method for administering the PI3K inhibitor is by injection and the method for administering the CD20 inhibitor is by injection.

In certain embodiments, a compound of Formula (I), or an isotopic variant thereof; or a pharmaceutically acceptable salt, solvate, hydrate, or prodrug thereof and a CD20 inhibitor is cyclically administered to a patient. As discussed above, cycling therapy involves the administration of an active agent or a combination of active agents for a period of time, followed by a rest for a period of time, and repeating this sequential administration. In some embodiments, cycling therapy reduces the development of resistance to one or more of the therapies, avoid or reduce the side effects of one of the therapies, and/or improves the efficacy of the treatment. In some embodiments, the compound of Formula (I) is administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the compound of Formula (I) is administered daily.

In some embodiments, the CD20 inhibitor is administered daily, every other day, every other day 3 times a week, every 3 days, every 4 days, every 5 days, every 6 days, weekly, every 2 weeks, every 3 weeks, every 4 weeks, every 5 weeks, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the CD20 inhibitor is administered 8 times in 6 months.

In some instances, the compound of Formula (I) or the CD20 inhibitor is optionally given continuously; alternatively, the dose of drug being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 8 days, 9 days, 10 days, 12 days, 14 days, 15 days, 20 days, 21 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In certain embodiments, in the treatment, prevention, or amelioration of one or more symptoms of the disorders, diseases, or conditions described herein, an appropriate dosage level of a compound of Formula (I), or an isotopic variant thereof; or a pharmaceutically acceptable salt, solvate, hydrate, or prodrug thereof generally is ranging from about 1 to 1000 mg, from about 1 to about 500 mg, from about 5 to about 500 mg, from about 5 to about 200 mg, from about 5 to about 250 mg or from about 10 to about 150 mg which can be administered in single or multiple doses. In certain embodiments, the compound of Formula (I), or an isotopic variant thereof; or a pharmaceutically acceptable salt, solvate, hydrate, or prodrug thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 450 or 500 mg. In certain embodiments, the compound of Formula (I), or an isotopic variant thereof; or a pharmaceutically acceptable salt, solvate, hydrate, or prodrug thereof is administered in an amount of about 60 mg, about 120 mg, about 150 mg, or about 180 mg. In certain embodiments, the compound of Formula (I), or an isotopic variant thereof; or a pharmaceutically acceptable salt, solvate, hydrate, or prodrug thereof is administered in an amount of about 60 mg. In certain embodiments, the compound of Formula (I), or an isotopic variant thereof; or a pharmaceutically acceptable salt, solvate, hydrate, or prodrug thereof is administered in an amount of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 225, about 250, about 275, about 300, about 325, about 350, about 375, about 400, about 450, or about 500 mg/day. In certain embodiments, the compound of Formula (I), or an isotopic variant thereof; or a pharmaceutically acceptable salt, solvate, hydrate, or prodrug thereof is administered in an amount of about 45 mg/day. In certain embodiments, the compound of Formula (I), or an isotopic variant thereof; or a pharmaceutically acceptable salt, solvate, hydrate, or prodrug thereof is administered in an amount of about 60 mg/day. In certain embodiments, the compound of Formula (I), or an isotopic variant thereof; or a pharmaceutically acceptable salt, solvate, hydrate, or prodrug thereof is administered in an amount of about 90 mg/day. In certain embodiments, the compound of Formula (I), or an isotopic variant thereof; or a pharmaceutically acceptable salt, solvate, hydrate, or prodrug thereof is administered in an amount of about 120 mg/day. In certain embodiments, the compound of Formula (I), or an isotopic variant thereof; or a pharmaceutically acceptable salt, solvate, hydrate, or prodrug thereof is administered in an amount of about 150 mg/day. In certain embodiments, the compound of Formula (I), or an isotopic variant thereof; or a pharmaceutically acceptable salt, solvate, hydrate, or prodrug thereof is administered in an amount of about 180 mg/day.

For oral administration, the pharmaceutical compositions provided herein can be formulated in the form of tablets containing from about 1.0 to about 1,000 mg of a compound of Formula (I), or an isotopic variant thereof; or a pharmaceutically acceptable salt, solvate, hydrate, or prodrug thereof, in one embodiment, about 1, about 5, about 10, about 15, about 20, about 25, about 50, about 75, about 100, about 150, about 200, about 250, about 300, about 400, about 500, about 600, about 750, about 800, about 900, and about 1,000 mg of the a compound of Formula (I), or an isotopic variant thereof; or a pharmaceutically acceptable salt, solvate, hydrate, or prodrug thereof for the symptomatic adjustment of the dosage to the patient to be treated.

In some embodiments, the pharmaceutical compositions provided herein can be formulated in the form of tablets containing about 45 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 45 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 45 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 45 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 45 mg daily until disease progression or intolerable toxicity.

In some embodiments, the pharmaceutical compositions provided herein can be formulated in the form of tablets containing about 60 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 60 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 60 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 60 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 60 mg daily until disease progression or intolerable toxicity.

In some embodiments, the pharmaceutical compositions provided herein can be formulated in the form of tablets containing about 90 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 90 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 90 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 90 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 90 mg daily until disease progression or intolerable toxicity.

In some embodiments, the pharmaceutical compositions provided herein can be formulated in the form of tablets containing about 120 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 120 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 120 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 120 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 120 mg daily until disease progression or intolerable toxicity.

In some embodiments, the pharmaceutical compositions provided herein can be formulated in the form of tablets containing about 150 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 150 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 150 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 150 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 150 mg daily until disease progression or intolerable toxicity.

In some embodiments, the pharmaceutical compositions provided herein can be formulated in the form of tablets containing about 180 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 180 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 180 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 180 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 180 mg daily until disease progression or intolerable toxicity.

In certain embodiments, the CD20 inhibitor used in combination with a compound of Formula (I), or an isotopic variant thereof; or a pharmaceutically acceptable salt, solvate, hydrate, or prodrug, is rituximab. In certain embodiments, the methods described herein further comprise administering rituximab as an intravenous infusion in 28 days cycles. In certain embodiments, rituximab is administered as an intravenous infusion for multiple 28 days cycles. In certain embodiments, rituximab is administered as an intravenous infusion at a dose of 375 mg/m² in the first cycle and 500 mg/m² in cycles 2-6. In certain embodiments, rituximab is administered intravenously as an infusion at a dose of 375 mg/m² per cycle. In certain embodiments, rituximab is administered as an intravenous infusion at a dose of 375 mg/m² for a total of 8 doses in 6 months. In certain embodiments, rituximab, or a variant or biosimilar thereof, is administered as an intravenous infusion at a dose of 375 mg/m² until disease progression or intolerable toxicity.

In certain embodiments, the CD20 inhibitor used in combination with a compound of Formula (I), or an isotopic variant thereof; or a pharmaceutically acceptable salt, solvate, hydrate, or prodrug, is ofatumumab. In certain embodiments, the methods described herein further comprise administering ofatumumab as an intravenous infusion every week. In certain embodiments, ofatumumab is administered as an intravenous infusion for multiple cycles. In certain embodiments, ofatumumab is administered as an intravenous infusion at a dose of 300 mg initial dose, followed 1 week later by 2,000 mg weekly for 7 doses, followed 4 weeks later by 2,000 mg every 4 weeks for 4 doses.

In certain embodiments, the CD20 inhibitor used in combination with a compound of Formula (I), or an isotopic variant thereof; or a pharmaceutically acceptable salt, solvate, hydrate, or prodrug, is obinutuzumab. In certain embodiments, the methods described herein further comprise administering obinutuzumab as an intravenous infusion in 28 days cycles. In certain embodiments, obinutuzumab is administered as an intravenous infusion for multiple cycles 28 days cycles. In certain embodiments, obinutuzumab is administered as an intravenous infusion at a dose of 100 mg on day 1 and 900 mg on day 2 Cycle 1, 1000 mg on day 8 and 15 of Cycle 1, and 1000 mg on day 1 of Cycles 2-6. In certain embodiments, obinutuzumab is administered as an intravenous infusion at a dose of 1000 mg on day 1, 8 and 15 of Cycle 1, and 1000 mg on day 1 of Cycles 2-6, and then every 2 months for 2 years.

In certain embodiments, the CD20 inhibitor used in combination with a compound of Formula (I), or an isotopic variant thereof; or a pharmaceutically acceptable salt, solvate, hydrate, or prodrug, is ocaratuzumab. In certain embodiments, the methods described herein further comprise administering ocaratuzumab as a subcutaneous injection every week. In certain embodiments, ocaratuzumab is administered as a subcutaneous injection for multiple cycles. In certain embodiments, ocaratuzumab is administered at a dose between about 20 mg to about 100 mg per week. In certain embodiments, ocaratuzumab is administered at a dose of about 40 mg per week. In certain embodiments, ocaratuzumab is administered at a dose of about 80 mg per week.

In certain embodiments, the CD20 inhibitor used in combination with a compound of Formula (I), or an isotopic variant thereof; or a pharmaceutically acceptable salt, solvate, hydrate, or prodrug, is ocrelizumab. In certain embodiments, the methods described herein further comprise administering ocrelizumab as an intravenous infusion in 24 weeks cycles. In certain embodiments, ocrelizumab is administered as an intravenous infusion for multiple cycles. In certain embodiments, ocrelizumab is administered as an intravenous infusion at a dose of 600 mg as a 300 mg infusion on days 1 and 15 for the first dose and as a single infusion of 600 mg for all subsequent infusions every 24 weeks.

In certain embodiments, the CD20 inhibitor used in combination with a compound of Formula (I), or an isotopic variant thereof; or a pharmaceutically acceptable salt, solvate, hydrate, or prodrug, is ublituximab. In certain embodiments, the methods described herein further comprise administering ublituximab as an intravenous infusion in cycles. In certain embodiments, ublituximab is administered as an intravenous infusion for multiple cycles. In certain embodiments, ublituximab is administered as an intravenous infusion at day 1, day 8 and day 15 of every cycle.

In certain embodiments, the CD20 inhibitor used in combination with a compound of Formula (I), or an isotopic variant thereof; or a pharmaceutically acceptable salt, solvate, hydrate, or prodrug, is BTCT4465A. In certain embodiments, the methods described herein further comprise administering BTCT4465A as an intravenous infusion in 21 days cycles. In certain embodiments, BTCT4465A is administered as an intravenous infusion for multiple cycles. In certain embodiments, BTCT4465A is administered as an intravenous infusion on Day 1 of each 21-day cycle. In certain embodiments, BTCT4465A is administered as an intravenous infusion on Days 1, 8, and 15 of Cycle 1 and thereafter on Day 1 of each 21-day cycle.

In certain embodiments, the CD20 inhibitor used in combination with a compound of Formula (I), or an isotopic variant thereof; or a pharmaceutically acceptable salt, solvate, hydrate, or prodrug, is veltuzumab. In certain embodiments, the methods described herein further comprise administering veltuzumab as an intravenous infusion or a subcutaneous injection in weekly cycles. In certain embodiments, veltuzumab is administered as an intravenous infusion or a subcutaneous injection for multiple weekly cycles. In certain embodiments, veltuzumab is administered as an intravenous infusion or a subcutaneous injection at a dose of 80 mg/m² once weekly for 4 weeks. In certain embodiments, veltuzumab is administered as an intravenous infusion or a subcutaneous injection at a dose of 120 mg/m² once weekly for 4 weeks. In certain embodiments, veltuzumab is administered as an intravenous infusion or a subcutaneous injection at a dose of 200 mg/m² once weekly for 4 weeks. In certain embodiments, veltuzumab is administered as an intravenous infusion or a subcutaneous injection at a dose of 375 mg/m² once weekly for 4 weeks.

In certain embodiments, the CD20 inhibitor used in combination with a compound of Formula (I), or an isotopic variant thereof; or a pharmaceutically acceptable salt, solvate, hydrate, or prodrug, is TRU-015. In certain embodiments, the methods described herein further comprise administering TRU-015 as an intravenous infusion in weekly cycles. In certain embodiments, TRU-015 is administered as an intravenous infusion for multiple weekly cycles. In certain embodiments, TRU-015 is administered at a dose between about 100 mg to about 1200 mg per week. In certain embodiments, TRU-015 is administered at a dose of about 400 mg per week. In certain embodiments, TRU-015 is administered at a dose of about 700 mg per week. In certain embodiments, TRU-015 is administered at a dose of about 1000 mg per week.

In certain embodiments, the CD20 inhibitor is administered weekly. In certain embodiments, the CD20 inhibitor is administered once every two, three, four, or five weeks. In certain embodiments, the CD20 inhibitor is administered once every four weeks. In certain embodiments, the CD20 inhibitor is administered is 21 days cycles. In certain embodiments, the CD20 inhibitor is administered is 28 days cycles. In certain embodiments, the CD20 inhibitor is administered intravenously. In certain embodiments, the CD20 inhibitor is administered as an intravenous infusion. In certain embodiments, the CD20 inhibitor is administered subcutaneously.

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Immunochemotherapy

Some embodiments provided herein describe treating patients with follicular lymphoma who have early disease progression after immuochemotherapy treatment. In some embodiments, the immunochemotherapy treatment is first-line treatment or first-line therapy. In some embodiments, the immunochemotherapy treatment is an additional (or subsequent) line of treatment or an additional (or subsequent) line of therapy. In some embodiments, the immunochemotherapy treatment is second-line treatment or second-line therapy. In some embodiments, the immunochemotherapy treatment is third-line treatment or third-line therapy. In some embodiments, the immunochemotherapy treatment is fourth-line treatment or fourth-line therapy. In some embodiments of the methods provided herein, the FU is relapsed/refractory FU. In some embodiments of the methods provided herein, the FU is relapsed/refractory FU after failure of at least two prior lines of systemic therapy in the subject. In some embodiments of the methods provided herein, the FU is relapsed/refractory FU after failure of at least two prior lines of systemic therapy in the subject, wherein the systemic therapy comprises an antiCD20 antibody and/or chemotherapy with an alkylating agent or a purine analogue. In some embodiments, the two prior lines of systemic therapy comprise an antiCD20 antibody and/or chemotherapy with an alkylating agent or a purine analogue.

In some embodiments, the immunochemotherapy treatment the patient previously received is i) BR (bendamustine and rituximab); ii) RCHOP (rituximab, cyclophosphamide, doxorubicin, vincristine, and prednisone); iii) RCVP (rituximab, cyclophosphamide, vincristine sulfate, prednisone); iv) FCMR (fludarabine, cyclophosphamide, mitoxantrone and rituximab); v) fludarabine and rituximab; vi) RFND (rituximab, fludarabine, mitoxantrone, dexamethasone); vii) R-MCP (rituximab with mitoxantrone, chlorambucil, and prednisolone); viii) R-FM (rituximab with fludarabine and mitoxantrone); ix) G-CHOP (Gazyza, cyclophosphamide, doxorubicin hydrochloride, vincristine sulfate, and prednisone); or a combination thereof.

In some embodiments, the immunochemotherapy treatment the patient previously received is i) BR (bendamustine and rituximab); ii) RCHOP (rituximab, cyclophosphamide, doxorubicin, vincristine, and prednisone); iii) RCVP (rituximab, cyclophosphamide, vincristine sulfate, prednisone); iv) FCMR (fludarabine, cyclophosphamide, mitoxantrone and rituximab); v) fludarabine and rituximab; vi) RFND (rituximab, fludarabine, mitoxantrone, dexamethasone); or a combination thereof.

In some embodiments, the immunochemotherapy treatment the patient previously received is i) RB (rituximab and bendamustine); ii) RCHOP (rituximab, cyclophosphamide, doxorubicin hydrochloride, vincristine sulfate, and prednisone); iii) RCVP (rituximab, cyclophosphamide, vincristine sulfate, prednisone); iv) FCMR (fludarabine, cyclophosphamide, mitoxantrone and rituximab); v) fludarabine and rituximab; vi) RFND (rituximab, fludarabine, mitoxantrone, dexamethasone); or a combination thereof.

In some embodiments, the immunochemotherapy treatment the patient previously received is RB (rituximab and bendamustine). In some embodiments, the immunochemotherapy the patient previously received is R-CHOP (rituximab, cyclophosphamide, doxorubicin, vincristine, and prednisone). In some embodiments, the immunochemotherapy treatment the patient previously received is RCVP (rituximab, cyclophosphamide, vincristine sulfate, prednisone). In some embodiments, the immunochemotherapy treatment the patient previously received is FCMR (fludarabine, cyclophosphamide, mitoxantrone and rituximab). In some embodiments, the immunochemotherapy treatment the patient previously received is fludarabine and rituximab. In some embodiments, the immunochemotherapy treatment the patient previously received is RFND (rituximab, fludarabine, mitoxantrone, dexamethasone). In some embodiments, the immunochemotherapy treatment the patient previously received is R-MCP (rituximab with mitoxantrone, chlorambucil, and prednisolone). In some embodiments, the immunochemotherapy treatment the patient previously received is R-FM (rituximab with fludarabine and mitoxantrone). In some embodiments, the immunochemotherapy treatment the patient previously received is G-CHOP (Gazyza, cyclophosphamide, doxorubicin hydrochloride, vincristine sulfate, and prednisone)

In some embodiments, the first-line immunochemotherapy treatment the patient previously received is RB (rituximab and bendamustine). In some embodiments, the first-line immunochemotherapy treatment the patient previously received is RCHOP (rituximab, cyclophosphamide, doxorubicin, vincristine, and prednisone). In some embodiments, the first-line immunochemotherapy treatment the patient previously received is RCVP (rituximab, cyclophosphamide, vincristine sulfate, prednisone). In some embodiments, the first-line immunochemotherapy treatment the patient previously received is FCMR (fludarabine, cyclophosphamide, mitoxantrone and rituximab). In some embodiments, the first-line immunochemotherapy treatment the patient previously received is fludarabine and rituximab. In some embodiments, the first-line immunochemotherapy treatment the patient previously received is RFND (rituximab, fludarabine, mitoxantrone, dexamethasone). In some embodiments, the first-line immunochemotherapy treatment the patient previously received is R-MCP (rituximab with mitoxantrone, chlorambucil, and prednisolone). In some embodiments, the first-line immunochemotherapy treatment the patient previously received is R-FM (rituximab with fludarabine and mitoxantrone).

In some embodiments, the additional (or subsequent) line of treatment the patient previously received is BR (bendamustine and rituximab). In some embodiments, the additional (or subsequent) line of treatment the patient previously received is RCHOP (rituximab, cyclophosphamide, doxorubicin, vincristine, and prednisone). In some embodiments, the additional (or subsequent) line of treatment the patient previously received is RCVP (rituximab, cyclophosphamide, vincristine sulfate, prednisone). In some embodiments, the additional (or subsequent) line of treatment the patient previously received is FCMR (fludarabine, cyclophosphamide, mitoxantrone and rituximab). In some embodiments, the additional (or subsequent) line of treatment the patient previously received is fludarabine and rituximab.

In some embodiments, the additional (or subsequent) line of treatment the patient previously received is RFND (rituximab, fludarabine, mitoxantrone, dexamethasone). In some embodiments, the additional (or subsequent) line of treatment the patient previously received is R-MCP (rituximab with mitoxantrone, chlorambucil, and prednisolone). In some embodiments, the additional (or subsequent) line of treatment the patient previously received is R-FM (rituximab with fludarabine and mitoxantrone).

In some embodiments, the immunochemotherapy treatment described above further comprises a BTK inhibitor. In certain embodiments, the BTK inhibitor is ibrutinib, BGB-3111, CC-292 (AVL-292), ACP 196 (Acalabrutinib), CNX-774, CGI1746, LFM-A13, CNX-774, ONO-4059, RN486 CPI-0610, DUAL946, GSK525762, I-BET151, JQ1, OTX015, PFI-1, RVX-208, RVX2135, or TEN-010, or a combination thereof. In certain embodiments, the BTK inhibitor is ibrutinib.

### Articles of Manufacture

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes one or more containers and a dosage form of a compound of Formula (I), or an isotopic variant thereof; or a pharmaceutically acceptable salt, solvate, hydrate, or prodrug thereof and a CD20 inhibitor.

In certain embodiments, the kit provided herein includes one or more containers and a dosage form of a compound of Formula (I), or an isotopic variant thereof; or a pharmaceutically acceptable salt, solvate, hydrate, or prodrug thereof and CD20 inhibitor. Kits provided herein can further include devices that are used to administer the active ingredients. Examples of such devices include, but are not limited to, syringes, needle-less injectors drip bags, patches, and inhalers.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### Examples

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL, (microliters); M (molar); mM (millimolar), μM (micro molar); eq. (equivalent); mmol (millimoles), Hz (Hertz), MHz (megahertz); hr or hrs (hour or hours); min (minutes); and MS (mass spectrometry).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies illustrated herein are intended to exemplify the applicable chemistry through the use of specific examples and are not indicative of the scope of the disclosure.

Syntheses of Compounds I-XVI are described in U.S. Pat. No. 9,056,852 B2, which is incorporated by reference for such disclosure.

### Example 1: Study of Treatment of Patients with Early Disease Progression

In an ongoing Phase 1b study, patients with relapsed B-cell malignancies are administered Compound A35 either as a single agent or in combination with the anti-CD20 antibody rituximab (Rituxan®). A total of 90 subjects have been enrolled in the study to date:
31 subjects enrolled in a dose escalation cohort of Compound A35 administered as a single agent at a daily dose of 60, 120, and 180 mg;
21 subjects enrolled in an ongoing expansion cohort of Compound A35 administered as a single agent at 60 mg/day, either on a daily continuous schedule (CS) only or on a daily dosing followed by a switch to an intermittent dosing schedule (SC) beginning in Cycle 3.
38 subjects enrolled in a cohort of Compound A35 at 60 mg/day on a CS or IS dosing plus infusional rituximab at a dose of 375 mg/m² administered for a total of 8 doses in 6 months

Information on 54 subjects with follicular lymphoma (FU) enrolled in the study are presented, of whom 30 (56%) were characterized as having POD24 (i.e., subjects with FL who experience progression of disease (POD) within 2 years (or 24 months) of initiating first-line immuno-chemotherapy with R-CHOP (rituximab, cyclophosphamide, doxorubicin, vincristine, and prednisone) or comparable regimens.

TABLE 1
Follicular Lymphoma Patient Characteristics

| | Compound | Compound | | | |
|---|---|---|---|---|---|
| | A35 Monotherapy | A35 + Rituximab | IS Group | CS Group | Total Patients |
| | (N = 40) | (N = 14) | (N = 23) | (N = 31) | (N = 54) |
| Age, in years | 66 (47-82) | 61 (38-81) | 61 (38-82) | 65 (47-81) | 63.5 (42-82) |
| Median (range) | 22 (55%) | 4 (29%) | 9 (39%) | 17 (55%) | 26 (48%) |
| No. subjects (%) | | | | | |
| ≥65 years | | | | | |
| Sex: Male/ | 67.5/32.5 | 78.6/21.4 | 69.6/30.4 | 71/29 | 70/30 |
| Female, % | | | | | |

Prior lymphoma therapy

| | | | | | |
|---|---|---|---|---|---|
| Median (range) | 2 (1-6) | 3 (1-10) | 2 (1-6) | 2 (1-10) | 2 (1-10) |
| number of prior | | | | | |
| therapies | | | | | |
| Patients with ≥2 | 25 (63%) | 10 (72%) | 18 (78%) | 17 (55%) | 35 (65%) |
| prior lines of therapy | | | | | |
| Prior anti-CD20 | 38 (95%) | 14 (100%) | 22 (95%) | 30 (97%) | 52 (96%) |
| antibody | | | | | |

### Efficacy Results in POD24

There was a very high rate of responses in the subset of FL subjects who were POD24 at enrollment, with a response rate comparable to that seen in the subset of subjects who were not POD24 at enrollment, as shown in Table 2. Only two POD24 subjects (both in the rituximab combination arm) did not achieve response during the study: one had progression of disease (POD) on Day 48 of therapy and one remained on stable disease (SD) during the study.

TABLE 2
Response Rate in all Efficacy Evaluable POD24 Patients

| | POD24 | | No POD24 | |
|---|---|---|---|---|
| | No. of | | No. of | |
| | Subjects | No. (%) of | Subjects | No. (%) of |
| | Evaluable for | Subjects with | Evaluable for | Subjects with |
| Cohort | Response | a Response | Response | a Response |
| A35 alone | 16 | 16 (100%) | 22 | 14 (64%) |
| A35 + rituximab | 10 | 8 (80%) | 2 | 2 (100%) |
| Total | 26 | 24 (92%) | 24 | 16 (67%) |

Of the other 24 POD24 subjects who achieve response, 6 had PD, 12 remain in the study, with a median follow-up of 8.5 months (range 3.4-25.8 months) from enrollment, and 6 have discontinued without evidence of PD at discontinuation, with a median follow-up in the study of 4.4 months (range, 3.0-8.8 months). The treatment with A35, alone or in combination with a CD20 inhibitor, achieves a very high rate of response in POD24 patients, which is higher than expected with conventional FL therapy.

### Example 2: Effect of Compound A35 and Rituximab on Treatment Efficacy in FL

Patients with FL were treated with Compound A35 either alone (N=40) or in combination with the anti-CD20 antibody rituximab (N=14). Most patients in the rituximab combination cohort received Compound A35 by intermittent schedule (IS) after 2 cycles of daily dosing of Compound A35.

Of the 14 subjects treated with Compound A35 and rituximab, 12 were evaluable for response and 10 achieved an objective response, as summarized in Table 2, which shows a high response rate with Compound A35 plus rituximab and Table 3, which shows a high rate of responses across all patient Groups.

TABLE 3
Disease Response Rate

| Patient Groups | Objective Responses*** |
|---|---|
| All FL patients | 40/50 (80%) |
| POD24 | 24/26 (92%) |
| Compound A35 alone | 30/38 (79%) |
| Compound A35 + rituximab | 10/12 (83%) |
| IS Group | 15/20 (75%) |
| CS Group | 25/30 (83%) |

***At least one post baseline assessment

### Example 3: Effect of Dosing Schedule of Compound A35 Alone or with Rituximab in Relapsed/Refractory (R/R) Follicular Lymphoma (FL)

Compound A35, a potent and selective oral PI3kδ inhibitor, is being evaluated in a Phase 1b study in patients (pts) with R/R B-cell malignancies.

Methods: Patients with ECOG ≤2, no prior PI3K therapy and progression of disease (POD) after ≥1 prior therapy were initially enrolled in a dose escalation phase (60-180 mg) then in 60 mg expansion cohorts as monotherapy or in combination with rituximab. Treatment is given in 28-day cycles and continued until POD or unacceptable toxicity. Compound A35 was given initially on a continuous daily dosing schedule. An intermittent dosing schedule on days 1-7 of a 28-day cycle was then evaluated after 2 cycles of daily dosing, or ≥3 cycles of daily dosing to reduce the risk of immune-related adverse events. For this analysis the intermittent schedule (IS) group is defined as patients who received Compound A35 alone or with rituximab daily for 2 cycles then switched to an intermittent schedule of 1 week per cycle, and the continuous schedule (CS) group is defined as patients who never switched to intermittent dosing or switched to intermittent dosing in cycle 4 or later cycles. Toxicity on CS is managed by a switch to IS. Progression of disease (POD) on IS is managed by a switch to CS.

Results: 58 FL patients received Compound A35 alone (n=40) or with rituximab (n=14). Median age 63.5 yrs. (range 48-82), median prior therapies 2 (range 1-10), 35 (65%) had ≥3 prior therapies and 27 (50%) were POD24. 32 patients (59%) remain on therapy at the time of the analysis with a follow-up ranging from 0.9 to 25.8 months, and 22 patients discontinued: 9 POD, 4 adverse events (AEs), 5 withdrew consent, and 4 to received a stem cell transplant. Grade 3 immune-related adverse events were less common in the IS group (N=23) compared to the CS group (N=31), as shown in Tables 4 and 5.

TABLE 4
Patients with Adverse Events of Special Interest

| | All Grades | | Grade 3 | |
|---|---|---|---|---|
| | CS Group | IS Group | CS Group | IS Group |
| AESI | (N = 31) | (N = 23) | (N = 31) | (N = 23) |
| Diarrhea/colitis | 11 (35.5%) | 11 (47.8%)* | 5 (16.1%) | 2 (8.7%) |
| Rash, all types | 11 (35.5%) | 2 (8.7%) | 4 (12.9%) | 0 |
| ALT/AST | 8 (25.8%) | 7 (30.4%) | 3 (9.7%) | 1 (4.3%) |
| increased | | | | |
| Pneumonia | 2 (6.5%) | 0 | 2 (6.5%) | 0 |
| Mucositis | 6 (19.4%) | 1 (4.3%) | 1 (3.2%) | 0 |

*Excluded 1 subject who had diarrhea after POD, and 1 subject who had diarrhea which only lasted one day with no dose change

TABLE 5
Patients with Adverse Events of Special Interest with Compound A35 Single Agent Therapy

| | All Grades | | | Related Grade 3 | | |
|---|---|---|---|---|---|---|
| | All | CS Group | IS Group | All | CS Group | IS Group |
| AESI | (N = 40) | (N = 25) | (N =15) | (N = 40) | (N = 25) | (N = 15) |
| Diarrhea/colitis | 17 (42.5%) | 10 (40.0%) | 7 (46.7%) | 6 (15.0%) | 5 (20.0%) | 1 (6.7%) |
| Rash, all types | 12 (30.0%) | 11 (44.0%) | 1 (6.7%) | 4 (10.0%) | 4 (16.0%) | 0 |
| ALT/AST | 9 (22.5%) | 6 (24.0%) | 3 (20.0%) | 3 (7.5%) | 3 (12.0%) | 0 |
| increased | | | | | | |
| Pneumonia/ | 2 (5.0%) | 2 (8.0%) | 0 | 2 (5.0%) | 2 (8.0%) | 0 |
| Pneumonitis | | | | | | |
| Mucositis | 6 (15.0%) | 6 (24.0%) | 0 | 1 (2.5%) | 1 (4.0%) | 0 |

*Excluded 1 subject who had diarrhea after POD, and 1 subject who had diarrhea which only lasted one day with no dose change

The rate of treatment discontinuation due to adverse events as also lower in the IS group than the CS group, as shown in Tables 6 and 7, indicating better tolerability of the IS regimen.

TABLE 6
Patient Dispositions on Intermittent or Continuous Schedule

| | CS Group | IS Group |
|---|---|---|
| Patient Disposition | (N = 31) | (N = 23) |
| Continuing on therapy | 14 (45%) | 18 (78%) |
| Discontinued therapy | 7 (23%) | 2 (9%) |
| Progressive disease | 4 (13%) | 0 (0%) |
| Adverse event | 3 (10%) | 1 (4%) |
| Stem cell transplant/ | 3 (10%) | 2 (9%) |
| Withdrawal of consent | | |
| Follow-up (months) | 6.5 (0.9-25.8) | 5.5 (0.9-15.5) |
| Median (range) | | |

TABLE 7
Patient Dispositions on Intermittent or Continuous
Schedule in Compound A35 Alone Arm

| | CS Group | IS Group |
|---|---|---|
| Patient Disposition | (N = 25) | (N = 15) |
| Continuing on therapy | 10 (40%) | 11 (73%) |
| Discontinued therapy | 5 (20%) | 3 (20%) |
| Progressive disease | 4 (16%) | 0 |
| Adverse event | 3 (12%) | 1 (7%) |
| Stem cell transplant | 3 (12%) | 0 |
| Withdrawal of consent | | |
| Follow-up (months) | 6.0 (0.9-25.8) | 5.5 (0.9-15.5) |
| Median (range) | | |

Objective responses in 40 of 50 patients (80%) with follow-up disease assessment: 79% with Compound A35 alone (including 20% morphologic/metabolic CR), 83% with Compound A35 plus rituximab, 92% in POD24, 75% in the IS group and 83% in the CS group.

The study results indicate that Compound A35 achieves a high rate of durable responses in R/R FL. IS appears to reduce the incidence of immune-related adverse events (irAEs) and maintains response to treatment. Progression of disease (POD) on IS can be salvaged by reverting to CS. A randomized study to evaluate Compound A35 given by IS or CS is enrolling pts with R/R FL, with switch to IS for irAEs and switch to CS if POD on IS.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating follicular lymphoma (FL), comprising administering to a subject in need thereof a therapeutically effective amount of a compound of Formula (I): 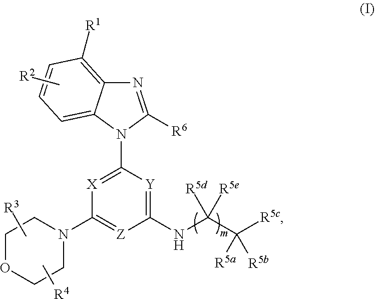
or an enantiomer, a mixture of enantiomers, a mixture of two or more diastereomers, or an isotopic variant thereof; or a pharmaceutically acceptable salt, solvate, or hydrate; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro;
(b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one or more, in one embodiment, one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein the subject has progression of disease within 24 months of initiating treatment of FL with immunochemotherapy (POD24) or the subject has relapsed/refractory FL.

2. The method of claim 1, or an enantiomer, a mixture of enantiomers, a mixture of two or more diastereomers, or an isotopic variant thereof; or a pharmaceutically acceptable salt, solvate, or hydrate; wherein R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

3. The method of claim 1, or an enantiomer, a mixture of enantiomers, a mixture of two or more diastereomers, or an isotopic variant thereof; or a pharmaceutically acceptable salt, solvate, or hydrate; wherein R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

4. The method of claim 3, or an enantiomer, a mixture of enantiomers, a mixture of two or more diastereomers, or an isotopic variant thereof; or a pharmaceutically acceptable salt, solvate, or hydrate; wherein R^{5a} and R^{5b} are each methyl, optionally substituted with one, two, or three halo(s).

5. The method of any one of claims 1-4, or an enantiomer, a mixture of enantiomers, a mixture of two or more diastereomers, or an isotopic variant thereof; or a pharmaceutically acceptable salt, solvate, or hydrate; wherein n is 1.

6. The method of any one of claims 1-5, or an enantiomer, a mixture of enantiomers, a mixture of two or more diastereomers, or an isotopic variant thereof; or a pharmaceutically acceptable salt, solvate, or hydrate; wherein R^{5f} and R^{5g} are each hydrogen.

7. The method of any one of claims 1-4, or an enantiomer, a mixture of enantiomers, a mixture of two or more diastereomers, or an isotopic variant thereof; or a pharmaceutically acceptable salt, solvate, or hydrate; wherein n is 0.

8. The method of any one of claims 1-7, or an enantiomer, a mixture of enantiomers, a mixture of two or more diastereomers, or an isotopic variant thereof; or a pharmaceutically acceptable salt, solvate, or hydrate; wherein m is 0.

9. The method of any one of claims 1-8, or an enantiomer, a mixture of enantiomers, a mixture of two or more diastereomers, or an isotopic variant thereof; or a pharmaceutically acceptable salt, solvate, or hydrate; wherein the compound of Formula (I) is of Formula (XI): 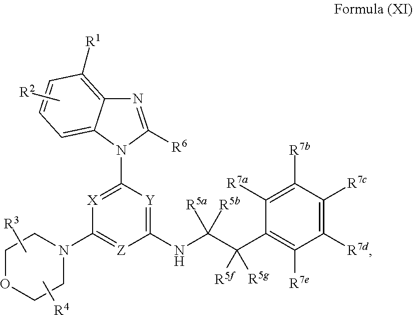
or an enantiomer, a mixture of enantiomers, a mixture of two or more diastereomers, or an isotopic variant thereof; or a pharmaceutically acceptable salt, solvate, hydrate, or prodrug thereof; wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or
two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

10. The method of claim 1, or an enantiomer, a mixture of enantiomers, a mixture of two or more diastereomers, or a pharmaceutically acceptable salt thereof;
wherein:
X, Y, and Z are each N;
R¹ and R² are each hydrogen;
R³ and R⁴ are each hydrogen;
R^{5a} is C₁₋₆ alkyl;
R^{5b} is C_{w} alkyl;
R^{5c} is —(CH₂)-phenyl, wherein R^{5c} is optionally substituted with one, two, three, or four substituents Q;
R^{5d} and R^{5e} are each hydrogen;
R⁶ is CHF₂;
m is 0; and
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

11. The method of claim 10, or an enantiomer, a mixture of enantiomers, a mixture of two or more diastereomers, or a pharmaceutically acceptable salt thereof; wherein R^{5a} and R^{5b} are each methyl, optionally substituted with one or more halos.

12. The method of any one of claims 1-11, or an enantiomer, a mixture of enantiomers, a mixture of two or more diastereomers, or a pharmaceutically acceptable salt thereof; wherein the compound of Formula (I) is Compound A35: 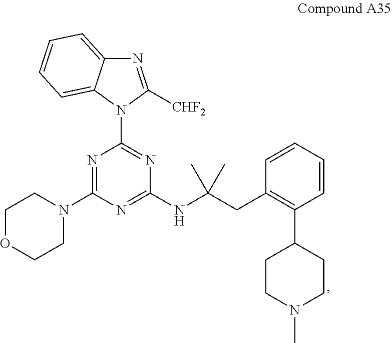 [SMILES: CN1CCC(c2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

13. The method of any one of claims 1-11, wherein the compound of Formula (I) is Compound A36: 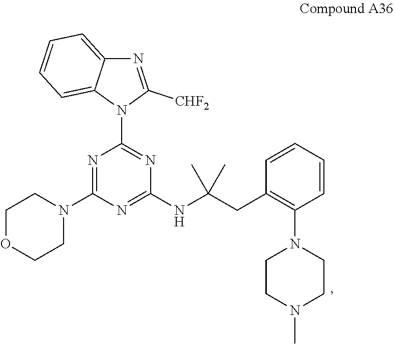 [SMILES: CN1CCN(c2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

14. The method of any one of claims 1-11, wherein the compound of Formula (I) is Compound A68: 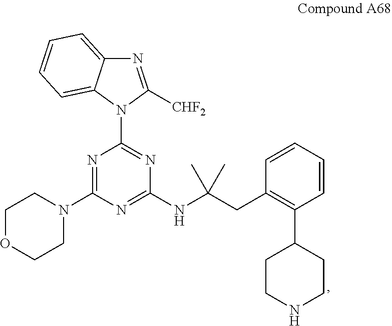 [SMILES: CC(C)(Cc1ccccc1C1CCNCC1)Nc1nc(N2CCOCC2)nc(-n2c(C(F)F)nc3ccccc32)n1]
or an isotopic variant thereof, a pharmaceutically acceptable salt, solvate, hydrate, or prodrug thereof.

15. The method of any one of claims 1-11, wherein the compound of Formula (I) is Compound A70: 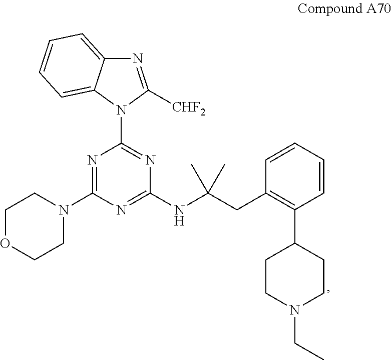 [SMILES: CCN1CCC(c2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

16. The method of any one of claims 1-11, wherein the compound of Formula (I) is Compound A37: 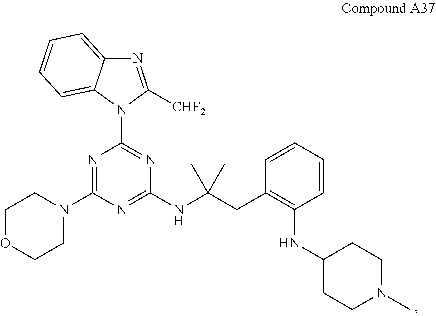 [SMILES: CN1CCC(Nc2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

17. The method of any one of claims 1-11, wherein the compound of Formula (I) is Compound A38: 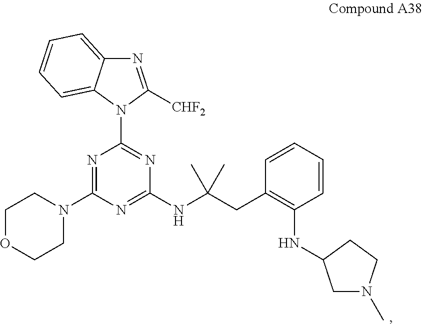 [SMILES: CN1CCC(Nc2ccccc2CC(C)(C)Nc2nc(N3CCOCC3)nc(-n3c(C(F)F)nc4ccccc43)n2)C1]
or an isotopic variant thereof, a pharmaceutically acceptable salt, solvate, hydrate, or prodrug thereof.

18. The method of any one of claims 1-11, wherein the compound of Formula (I) is Compound A41: 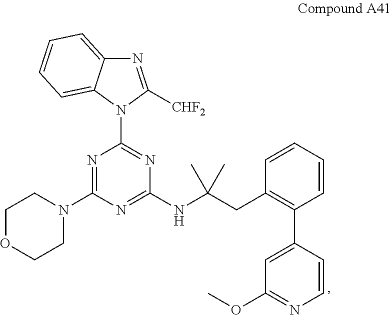 [SMILES: COc1cc(-c2ccccc2CC(C)(C)Nc2nc(N3CCOCC3)nc(-n3c(C(F)F)nc4ccccc43)n2)ccn1]
or an isotopic variant thereof, a pharmaceutically acceptable salt, solvate, hydrate, or prodrug thereof.

19. The method of any one of claims 1-11, wherein the compound of Formula (I) is Compound A42: 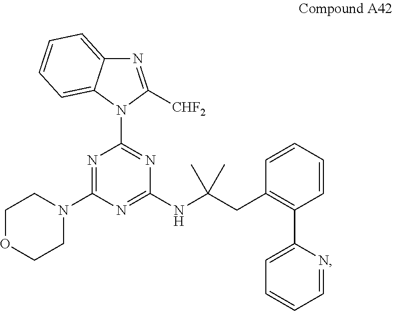 [SMILES: CC(C)(Cc1ccccc1-c1ccccn1)Nc1nc(N2CCOCC2)nc(-n2c(C(F)F)nc3ccccc32)n1]
or an isotopic variant thereof, a pharmaceutically acceptable salt, solvate, hydrate, or prodrug thereof.

20. The method of any one of claims 1-11, wherein the compound of Formula (I) is Compound A43: 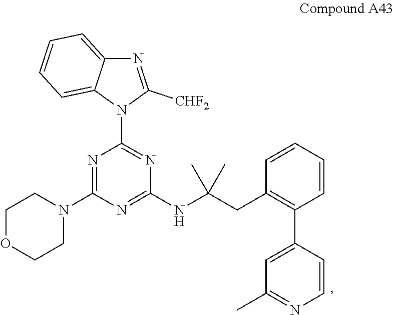 [SMILES: Cc1cc(-c2ccccc2CC(C)(C)Nc2nc(N3CCOCC3)nc(-n3c(C(F)F)nc4ccccc43)n2)ccn1]
or an isotopic variant thereof, a pharmaceutically acceptable salt, solvate, hydrate, or prodrug thereof.

21. The method of any one of claims 1-11, wherein the compound of Formula (I) is Compound A44: 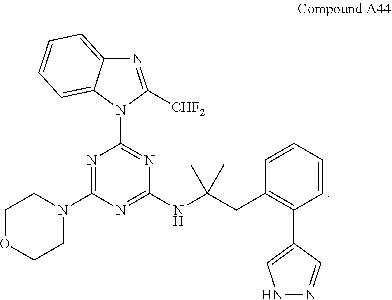 [SMILES: CC(C)(Cc1ccccc1-c1cn[nH]c1)Nc1nc(N2CCOCC2)nc(-n2c(C(F)F)nc3ccccc32)n1]
or an isotopic variant thereof, a pharmaceutically acceptable salt, solvate, hydrate, or prodrug thereof.

22. The method of any one of claims 1-21, wherein about 30 mg, about 60 mg, about 120 mg, or about 180 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

23. The method of any one of claims 1-22, wherein about 60 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

24. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject orally.

25. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is formulated as a tablet or capsule.

26. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject daily.

27. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day, twice per day, or three times per day.

28. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day.

29. The method of any one of the preceding claims, wherein about 60 mg/day of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

30. The method of any one of the preceding claims, wherein the subject has progression of disease within 24 months of initiating treatment of FL with first-line immunochemotherapy.

31. The method of any one of the preceding claims, wherein the subject has POD24 and the immunochemotherapy comprises administering to the subject a combination of chemotherapy and immunotherapy agents selected from i) RB (rituximab and bendamustine); ii) RCHOP (rituximab, cyclophosphamide, doxorubicin hydrochloride, vincristine sulfate, and prednisone); iii) RCVP (rituximab, cyclophosphamide, vincristine sulfate, prednisone); iv) FCMR (fludarabine, cyclophosphamide, mitoxantrone and rituximab); v) fludarabine+rituximab; vi) RFND (rituximab, fludarabine, mitoxantrone, dexamethasone); and vii) GCHOP (Gazyza, cyclophosphamide, doxorubicin hydrochloride, vincristine sulfate, and prednisone).

32. The method of any one of the preceding claims, wherein the immunochemotherapy comprises administering to the subject RCHOP (rituximab, cyclophosphamide, doxorubicin hydrochloride, vincristine sulfate, and prednisone).

33. The method of any one of the preceding claims, wherein the immunochemotherapy further comprises administering to the subject a BTK inhibitor.

34. The method of any one of the preceding claims, wherein the method comprises a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle.

35. The method of claim 34, wherein the CS is continued until at least one incidence of intolerable toxicity occurs/is observed.

36. The method of claim 35, wherein the at least one toxicity is enterocolitis, a cutaneous toxicity, liver toxicity, pulmonary toxicity, infection, or any combination thereof.

37. The method of any one of claims 1-33, wherein the method comprises at least three 28-day cycles, wherein:
(iii) the first two cycles comprise a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle; and
(iv) the third and subsequent cycles comprise an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle.

38. The method of claim 37, wherein T-cells are recovered and/or re-populated during the 21 days without treatment.

39. The method of claim 37 or 38, wherein regulatory T-cells (TREG) and/or effector T-cells are recovered and/or re-populated during the 21 days without treatment.

40. The method of any one of claims 37-39, wherein the incidence of at least one toxicity is reduced.

41. The method of claim 40, wherein the at least one toxicity is enterocolitis, a cutaneous toxicity, liver toxicity, pulmonary toxicity, infection, or any combination thereof.

42. The method of any one of claims 37-41, wherein the IS is continued until progression of disease.

43. The method of any one of claims 1-42, wherein the subject has progression of disease within 24 months (POD24) of initiating treatment of FL with first-line immunochemotherapy, and wherein the immunochemotherapy comprises administering to the subject RCHOP (rituximab, cyclophosphamide, doxorubicin hydrochloride, vincristine sulfate, and prednisone).

44. The method of any one of the preceding claims, wherein an additional therapeutic agent is administered in combination with the compound of Formula (I), or an enantiomer, a mixture of enantiomers, a mixture of two or more diastereomers, or an isotopic variant thereof; or a pharmaceutically acceptable salt, solvate, hydrate, or prodrug thereof.

45. The method of claim 44, wherein the additional therapeutic agent is rituximab.

46. The method of claim 45, wherein rituximab is administered at a dose of about 375 mg/m².

47. The method of claim 45 or 46, wherein 8 doses of rituximab are administered to the subject over a period of about 6 months.

48. The method of any one of the preceding claims, wherein the subject is treated for a period of about 6 months.
